(19) United States
(12) Patent Application Publication
(10) Pub. No.: US 2004/0259244 A1
(43) Pub. Date: Dec. 23, 2004

(54) Methods, compositions, and growth and differentiation factors for insulin-producing cells

(76) Inventors: David William Scharp, Mission Viejo, CA (US); Paul Presley Latta, Irvine, CA (US); Margaret Coutts, Irvine, CA (US); Catherine Anne McIntyre, Aliso Viejo, CA (US); Sharon C. Presnell, Raleigh, NC (US); Mohammad A. Heidaran, Cary, NC (US); Perry D. Haaland, Chapel Hill, NC (US)

Correspondence Address: KNOBBE MARTENS OLSON & BEAR LLP, 2040 MAIN STREET, FOURTEENTH FLOOR, IRVINE, CA, 92614, US

(21) Appl. No.: 10/800,813

(22) Filed: Mar. 15, 2004

Related U.S. Application Data
(63) Continuation of application No. 10/447,319, filed on May 28, 2003.
(60) Provisional application No. 60/384,000, filed on May 28, 2002.

(51) Int. Cl. C12N 5/08

(52) U.S. Cl. 435/366

(57) ABSTRACT

A method of converting differentiated non-hormone producing pancreatic cells into differentiated hormone producing cells is disclosed. The method comprises two steps: first, culturing cells under conditions which convert differentiated non-hormone producing cells into stem cells; and second, culturing stem cells under conditions which provide for differentiating stem cells into hormone-producing cells. The invention defines growth and differentiation factors that are presented to the stem cells to result in their differentiation into hormone-producing cells, especially insulin-producing cells. The invention provides a new source of large quantities of hormone producing cells such as insulin-producing cells that are riot currently available for therapeutic uses such as the treatment of diabetes.

### RELATED APPLICATIONS

This application is a continuation of U.S. application Ser. No. 10/447,319, filed May 28, 2003 which claims priority to U.S. Provisional Application No. 60/384,000, filed May 28, 2002 which are both incorporated herein by reference in their entirety.

### FIELD OF THE INVENTION

This invention relates to the culture media, mode, conditions, and methods for converting non-insulin producing pancreas cells into stem cells that can be proliferated and differentiated into pancreatic hormone producing cells.

### DESCRIPTION OF THE RELATED ART

The ability to selectively control the in vitro expansion and conversion of non-insulin producing pancreatic cells, such as acinar cells or duct cells, into insulin producing cells, would create a new treatment regime for diabetes that avoids many of the shortcomings of current diabetes treatments.

Diabetes mellitus is a disease caused by the loss of the ability to transport glucose into the cells of the body, either because not enough insulin is produced or because the response to insulin is diminished. In a healthy person, minute elevations in blood glucose stimulate the production and secretion of insulin, the role of which is to increase glucose uptake into cells, returning the blood glucose to the optimal level. Insulin stimulates liver and skeletal muscle cells to take up glucose from the blood and convert it into the energy storage molecule glycogen. It also stimulates skeletal muscle fibers to take up amino acids from the blood and convert them into protein, and it acts on adipose (fat) cells to stimulate the synthesis of fat. In diabetes, the blood stream may be saturated with glucose, but the glucose cannot reach the intracellular places where it is needed and utilized. As a result the cells of the body are starved of needed energy, which leads to the wasted appearance of many patients with poorly controlled insulin-dependent diabetes.

Prior to the discovery of insulin and its use as a treatment for diabetes, the only outcome was starvation followed predictably by death. With insulin treatment today, death still occurs with over dosage of insulin resulting in extreme hypoglycemia and coma followed by death unless reversed by the intake of glucose. Death also still occurs with major under dosage of insulin leading to ketoacidosis that, if not treated properly and urgently will also result in coma and death.

While diabetes is not commonly a fatal disease thanks to the treatments available to diabetics today, none of the standard treatments can replace the body's minute-to-minute production of insulin and precise control of glucose metabolism. As a consequence, the average blood glucose levels in diabetics remain generally too high. The chronically elevated blood glucose levels cause a number of long-term complications over time. Diabetes is the leading cause of blindness, renal failure, the premature development of heart disease or stroke, gangrene and amputation, impotence, and it decreases the sufferer's overall life expectancy by one to two decades.

Diabetes mellitus is one of the most common chronic diseases in the world. In the United States, diabetes affects approximately 16 million people—more than 12% of the adult population over 45. The number of new cases is increasing by about 150,000 per year. In addition to those with clinical'diabetes, there are approximately 20 million people showing symptoms of abnormal glucose tolerance. These people are borderline diabetics, midway between those who are normal and those who are clearly diabetic. Many of them will develop diabetes in time and some estimates of the potential number of diabetics are as high as 36 million or 25-30% of the adult population over 45 years.

Diabetes and its complications have a major socioeconomic impact on modern society. Of the approximately $700 billion dollars spent on healthcare in the US today, roughly $100 billion are spent to treat diabetes and its complications. Since the incidence of diabetes is rising, the costs of diabetes care will occupy an ever-increasing fraction of total healthcare expenditures unless steps are taken promptly to meet the challenge. The medical, emotional and financial toll of diabetes is enormous, and increases as the numbers of those suffering from diabetes grows.

Diabetes mellitus can be subdivided into two distinct types: Type 1 diabetes and Type 2 diabetes. Type 1 diabetes is characterized by little or no circulating insulin and it most commonly appears in childhood or early adolescence. It is caused by the destruction of the insulin-producing beta cells of the pancreatic islets. There is a genetic predisposition for Type 1 diabetes with the destruction resulting from an autoimmune attack against the beta cells, initiated by some as yet unidentified environmental event, such as a viral infection, or the action of a noninfectious agent (a toxin or a food), which triggers the immune system to react to and destroy the patient's beta cells in the pancreas. The pathogenic sequence of events leading to Type 1 diabetes is thought to consist of several steps. First, it is believed that genetic susceptibility is an underlying requirement for the initiation of the pathogenic process. Secondly, an environmental insult mediated by a virus or noninfectious agent such as toxin or food triggers the third step, the inflammatory response in the pancreatic islets (insulitis) in genetically predisposed individuals. The fourth step is an alteration or transformation of the beta cells such that they are no longer recognized as “self” by the immune system, but rather seen as foreign cells or “nonself”. The last step is the development of a full-blown immune response directed against the “targeted” beta cells, during which cell-mediated immune mechanisms cooperate with cytotoxic antibodies in the destruction of the insulin-producing beta cells. Despite this immune attack, for a period of time, the production of new beta cells is fast enough to stay ahead of the destruction by the immune system and a sufficient number of beta cells are present to control blood glucose levels. Gradually, however, the number of beta cells declines. When the number of beta cells drops to a critical level (10% of normal), blood glucose levels can no longer be controlled and the progression to total failure of insulin production is almost inevitable. It is thought that the regeneration of beta cells continues for a few years, even after functional insulin production ceases, but that the cells are destroyed as they develop maturity.

To survive, people with Type 1 diabetes must take multiple insulin injections daily and test their blood sugar by pricking their fingers for blood multiple times per day. The multiple daily injections of insulin do not adequately mimic the body's minute-to-minute production of insulin and precise control of glucose metabolism. Blood sugar levels are usually higher than normal, causing complications that include blindness, heart attack, kidney failure, stroke, nerve damage, and amputations. Even with insulin, the average life expectancy of a diabetic is 15-20 years less than that of a healthy person.

Type 2 diabetes usually appears in middle age or later and particularly affects those who are overweight. Over the past few years, however, the incidence of Type 2 diabetes mellitus in young adults has increased dramatically. In the last several years, the age of onset of Type 2 diabetes has dropped from 40 years of age to 30 years of age with those being obese, the new younger victims of this disease. In Type 2 diabetes, the body's cells that normally require insulin lose their sensitivity and fail to respond to insulin normally. This insulin resistance may be overcome for many years by extra insulin production by the pancreatic beta cells. Eventually, however, the beta cells are gradually exhausted because they have to produce large amounts of excess insulin due to the elevated blood glucose levels. Ultimately, the overworked beta cells die and insulin secretion fails, bringing with it a concomitant rise in blood glucose to sufficient levels that it can only be controlled by exogenous insulin injections. High blood pressure and abnormal cholesterol levels usually accompany Type 2 diabetes. These conditions, together with high blood sugar, increase the risk of heart attack, stroke, and circulatory blockages in the legs leading to amputation. Drugs to treat Type 2 diabetes include some that act to reduce glucose absorption from the gut or glucose production by the liver and others that stimulate the beta cells directly to produce more insulin. However, high levels of glucose are toxic to beta cells, causing a progressive decline of function and cell death. Consequently, many patients with Type 2 diabetes eventually need exogenous insulin. A recent disturbing finding is the increase in the estimate from 20% to 40% of the Type 2 diabetics that will eventually require insulin treatment.

Another form of diabetes is called Maturity Onset Diabetes of the Young (MODY). This form of diabetes is due to a genetic error in the insulin-producing cells that restricts its ability to process the glucose that enters this cell via a special glucose receptor. Beta cells in patients with MODY cannot produce insulin correctly in response to glucose, resulting in hyperglycemia and require treatment that eventually also requires insulin injections.

The currently available medical treatments for insulin-dependent diabetes are limited to insulin administration and pancreas transplantation either with whole pancreas or pancreas segments. Insulin therapy is by far more prevalent than pancreas transplantation and entails administration of insulin either conventionally, by multiple subcutaneous injections, or by continuous subcutaneous injections. Conventional insulin therapy involves the administration of one or two injections a day of intermediate-acting insulin with or without the addition of small amounts of regular insulin. The multiple subcutaneous insulin injection technique involves administration of intermediate- or long-acting insulin in then evening and/or morning as a single dose together with regular insulin prior to each meal. Continuous subcutaneous insulin infusion involves the use of a small battery-driven pump that delivers insulin subcutaneously to the abdominal wall, usually through a 27-gauge-butterfly needle. With this treatment modality, insulin is delivered at a basal rate continuously throughout the day and night, with increased rates programmed prior to meals. In each of these methods, the patient is required to frequently monitor his or her blood glucose levels and adjust the insulin dose if necessary. However, controlling blood sugar is not simple. Despite rigorous attention to maintaining a health diet, exercise regimen, and always injecting the proper amount of insulin, many other factors can adversely affect a person's blood-sugar control including: Stress, hormonal changes, periods of growth, illness or infection and fatigue. People with Type 1 diabetes must constantly be prepared for life threatening hypoglycemic (low blood sugar) and hyperglycemic (high blood sugar) reactions. Insulin-dependent diabetes is a life threatening disease which requires never-ending vigilance.

In contrast to insulin administration, whole pancreas transplantation or transplantation of segments of the pancreas is known to have cured diabetes in patients. However, due to the requirement for life-long immunosuppressive therapy, the transplantation is usually performed only when kidney transplantation is required, making pancreas-only transplantations relatively infrequent operations. Although pancreas transplants are very successful in helping people with insulin-dependent diabetes improve their blood sugar to the point they no longer need insulin injections and reduce long-term complications, there are a number of drawbacks to whole pancreas transplants. Most importantly, getting a pancreas transplant involves a major operation and requires the use of life-long immunosuppressant drugs to prevent the body's immune system from destroying the pancreas that is a foreign graft. Without these drugs, the pancreas is destroyed in a matter of days. The risks in taking these immunosuppressive drugs is the increased incidence of infections and tumors that can both be life threatening in their own right. The risks inherent in the operative procedure, the requirement for life-long immunosuppression of the patient to prevent rejection of the transplant and the morbidity and mortality rate associated with this invasive procedure, illustrate the serious disadvantages associated with whole pancreas transplantation for the treatment of diabetes. Thus, an alternative to both insulin injections and pancreas transplantation would fulfill a great public health need.

Islet transplants are much simpler (and safer) procedures than whole pancreas transplants and can achieve the same effect by replacing lost beta cells. Insulin producing beta cells are found in the islets of Langerhans scattered throughout the pancreas, an elongated gland located transversely behind the stomach. The pancreas secretes between 1.5 and 3 liters of alkaline fluid containing enzymes and pro-enzymes for digestion into the common bile duct. Histologically, the pancreas is composed of three types of functional cells: a) exocrine cells that secrete their enzymes into a lumen, b) ductal cells that carry the enzymes to the gut, and c) endocrine cells that secrete their hormones into the bloodstream. The exocrine portion is organized into numerous small glands (acini) containing columnar to pyramidal epithelial cells known as acinar cells. Acinar cells comprise approximately 80% of the pancreatic cells and are responsible for secreting digestive enzymes, such as amylases, lipases, phospholipases, trypsin, chymotrypsiri, aminopeptidases, elastase and various other proteins into the pancreatic duct system. The pancreatic duct system consists of an intricate, tributary-like network of interconnecting ducts that drain each secretory acinus, draining into progressively larger ducts, and ultimately draining into the main pancreatic duct. The lining epithelium of the pancreatic duct system consists of duct cells, a cell type comprising approximately 10% of pancreatic cells. Duct cell morphology ranges from cuboidal in the fine radicles draining the secretory acini to tall, columnar, mucus-secreting in the main ductal system.

The endocrine portion of the pancreas is composed of about 1 million small endocrine glands, the islets of Langerhans, scattered throughout the exocrine pancreas. Although the islet cells comprise only approximately 2% of the pancreatic cells, the islet cells are responsible for the maintenance of blood glucose levels by secreting insulin appropriately and are the most important cells in the pancreas. There are seven types of islet cells classified according to the type of endocrine hormone secreted. The beta cells of the islet produce insulin. As discussed above, when there are insufficient numbers of beta cells, or insufficient insulin secretion, regardless of the underlying reason, diabetes results. Reconstituting the islet beta cells in a diabetic patient to a number sufficient to restore normal glucose-responsive insulin production would solve the problems associated with both insulin injection and major organ transplantation.

The islet transplantation outpatient procedure allows patients to remain fully conscious under local anesthesia while the equivalent of a 2-3 milliliters of pure islet cells is piped through a small catheter to the liver. The patients can return home or to regular activities soon after the procedure. Thus, transplanting islets instead of transplanting the entire pancreas or segments thereof offers a number of ways around the risks of the whole organ transplant. However, the shortage of islet cells available for transplantation remains an unsolved problem in islet cell transplantation. Since islets form only about 2% of the entire pancreas, isolating them from the rest of the pancreas that does not produce insulin takes approximately 6 hours. Although an automated isolation method has made it possible to isolate enough islets from one pancreas to transplant into one patient, as opposed to the 5 or 6 organs previously needed to carry out one transplant, the demand for islets still exceeds the currently available supply of organs harvested from cadavers. In the United States, due to a combination of low organ donor rates and the increasing occurrence of insulin-dependent diabetes, there are only approximately 6,000 pancreases available for transplantation or islet cell isolation, while the new cases of insulin-dependent diabetes diagnosed each year number approximately 35,000 (Hering, B. J. & Ricordi, C. (1999) Graft 2, 12-27).

One solution to the problem of severe islet cell shortage is the genetic engineering of other cells to produce insulin. Genetically engineering other cells to produce insulin has already shown some success in muscle and liver cells in that they can be modified to produce proinsulin, the precursor to insulin. However, improving secretion of the insulin in these genetically engineered cells will still require considerable investigative effort and their low insulin production renders them as yet unsuitable for transplantation. Another strategy, xenotransplantation, the transplant of an organ (or tissues or cells, in the case of diabetes) from one species to another faces a number of fundamental obstacles to becoming a viable alternative to insulin injections of human transplantation. The risks associated with xenotransplantation include transfer of prions such as those causing mad cow disease (bovine spongiform encephalopathy or BSE), and transmission of animal retroviruses such as PoERV (porcine endogenous retrovirus). Another obstacle is the problem of hyperacute rejection. The more distant the two species involved in the transplant are in evolutionary terms, the more rapid and severe the rejection process when the organs of one are transplanted into the other and the need for stronger and more risky immuno suppression. Strategies involving the genetic engineering of animal islets so as to make them less likely to succumb to immune system attach and destruction poses the risk of tampering with the silent human endogenous retroviral sequences (HERVs) thousands of which are spread throughout the human genome. Activation of these sequences by recombination and the ensuing expression of HERV proteins may lead to cancer or immune system dysregulation (Romano et al., Stem Cells 2000, 18:19-39). Finally, animal and human organs and cells differ in many ways: In their anatomy or structure, production of hormones, rates of filtration, secretion and absorption of enzymes and other chemicals, in their resistance to disease, and expected longevity.

Another strategy to solve the problem of tissue availability for islet cell transplantation is the isolation of embryonic or totipotent stem cells. Totipotent stem cells are cells that are capable of growing into any other type of cell in the body, including into an entire organism. The problem with using this type of stem cell to grow as many islets as are needed to meet the demand for transplants for diabetes lies in their procurement from abortions or in vitro fertilizations with inherent ethical and political risks. Furthermore, the techniques to differentiate totipotent stem cells into normal insulin-producing cells has not been perfected and controlled in terms of their routine differentiation into insulin-producing cells in the great quantities that will be needed. Their ability to produce insulin in response to increases in glucose concentration that trigger insulin secretion in normal beta cells, indicating that they are not behaving as normal islet beta cells (Vogel, Science, 2001 292: 615-617). Finally, the use of embryonic stem cells for therapeutic purposes in patients carries the inherent danger of tumor growth. Mouse embryonic stem cells are tumorigenic when injected into adult mice, and human embryonic stem cells also demonstrate a similar tumorigenic potential when injected into immune incompetent mice. The potential use of embryonic stem cells requires the precise separation of undifferentiated stem cells from the desired differentiated progeny, a critical and as yet unattained prerequisite for clinical application (Solter and Gearhart, Science 1999, 283: 1468-1470) in order to prevent potential tumor formation.

Thus, there exists a critical unmet medical need for large numbers of non-tumorigenic human beta cells to treat millions of diabetic patients worldwide. A strategy for the large-scale production of human insulin-producing beta cells from readily available starting material such as pancreatic acinar and duct cells that are converted into clinically relevant stem cells, would overcome the obstacles faced by the current approaches.

In examining the prior art in terms of beginning with primary pancreatic cells and converting them to insulin producing cells, the experience historically falls into three categories based on the starting cells of interest: either islet cells, duct cells, or acinar cells. There are many prior experiences starting with islet cells to grow and expand the islet cell mass in vitro. Essentially all of these approaches isolate purified islets and place them predominantly into adherent culture systems in which the islets loose their islet phenotype, plate out as single cells, and grow to confluence. Most efforts to induce direct differentiated islet cell replication in vitro have shown limited capability to proliferate islet cell mass while maintaining their differentiated state. The collected experience of these studies is that in most circumstances, after a period of culture of these adherent islet cells, they lose their islet phenotype and dedifferentiate into a more primitive cell type that is poorly characterized but expands for a time in vitro. Yet, these cells invariably enter into senescence with the loss of the cultures.

It has proved very difficult to redifferentiate these more primitive cells back to differentiated islets (Nielson 92, Brelje 93, Bonner Weir 93, Otonkoski 91, Otonkoski 94). However, in one approach (Cornelius 97), the islet cultures from NOD mice were allowed to plate and then were left without media changes for several weeks. A few cells of a poorly identified epithelial cell type was all that survived and could be grown out that demonstrated the ability to proliferate and could be differentiated into islet cells with different stages of culture conditions and reagents. The resulting U.S. Pat. Nos. 5,834,308 and 6,001,647, claim these poorly described epithelial cells as stem cells that require this method of culture to isolate, grow, and develop them into functional insulin-producing cells. While demonstrating the presence of stem cells by this method of pancreatic cell adherent culture, the technique of starvation of the cells to a minimal survival, and growth and differentiation into islet cells is problematic. This approach requires extensive growth of islet cells to reach the levels required to produce large scale implants for the treatment of diabetes. There is no evidence to date that this procedure is applicable to human cells and that such a scale up is possible while retaining the differentiated phenotype of these islet cells required for a clinical product. Therefore, we have turned to an alternative approach as described in this invention that significantly differs in that it does not start with primary islet cells to form the stem cells that can be expanded and differentiated to insulin producing cells. Instead, we start with non-insulin-producing pancreatic cells, and convert them to stem cells that expand and then differentiate into islet cells.

Others have placed the islet cells into MATRIGEL, collagen, or agarose rather than the use adherent cultures (Kerr-Conte 96). This results in the formation of cystic duct structures with regression of islet tissues and growth and differentiation of duct structures and cells of ductal phenotype. The inventors of this application have also placed isolated human islets into MATRIGEL and have confirmed the induction of duct cells that replace the differentiated islet cell mass. Different matrices can also convert islet cells to duct cells, especially in the presence of HGF (Lefrbvre 98), but again fail to produce islets. While claims of islet cells forming from these structures have been made, it is unclear as to whether their origin is from residual islet tissue present in the starting cells or new insulin-producing cells. The duct structures and islet cells may also develop from a stem cell that has not as yet been specifically identified.

The next approach that has been explored is to start with pancreatic duct cells to determine the ability to form new islet cells. It is based on the observations in both developing fetal pancreas as well as adult pancreas induced to damage by disease or manipulation where one observes the formation of new islets budding off ductal structures that have led to the idea that there is a pancreatic stem cell associated with the ductal structures that can be activated by fetal development, or damage or loss to islet mass in the adult pancreas.

Starting from isolated and purified duct structures from mouse and rat pancreas and not from human pancreas (Fung, U.S. Pat. No. 6,326,201), single cells begin to form monolayers in vitro that are predominantly a mixture of fibroblasts and stromal cells. Eventually some insulin producing cells begin to appear in these adherent cultures, but remain at a low level in the monolayers. Addition of a few growth factors minimally increased numbers of insulin cells in the monolayer. But, single cells to groups of cells, called non-adhering cells (NAC) began to appear floating above the monolayer cultures that contain islet hormone cell types. These NAC's could be increased by using growth factor pulsing prior to harvesting. They also described pdx1 positive cells, some costaining with insulin which is required as a beta cell, and others with pdx1 staining only that they describe as being progenitor cells. The NAC's were also able to show glucose stimulated insulin release. They can also add different growth factors to the monolayers and induce proliferation as well as phenotype changes. They describe the use of lectins to purify these progenitor cells as they are produced. Thus, their results support the ability of purified pancreatic duct cells from large pancreatic ducts to be dedifferentiated into progenitor cells that can differentiate into insulin producing cells by the use of their specific methods. This invention differs significantly from the Fung work in that our starting pancreatic cells are human pancreatic cells and are not isolated from purified duct structures. In fact, he claims producing duct cells only from pancreatic duct tissue that he defines as including the main pancreatic duct, the accessory pancreatic duct, the dorsal pancreatic duct, and the ventral pancreatic duct. He separately defines interlobular ducts and intercalated ducts as separate entities that are not included in his definition of pancreatic duct. Our starting pancreatic tissue excludes the tissue he defines as pancreatic duct since these larger structures and parts of structures are screened out of our preparation during the cell isolation process and are not observed in the histologic sections of the starting material. The only pancreatic duct tissue staining positive for CK19 are the intercalated ducts located within acinar cell aggregates and completely surrounded by acinar cells.

Thus, our starting pancreatic cells are a mixture of acinar cells, intercalated duct cells surrounded by acinar cells, and stromal cells, that are harvested after purifying the islets out of the starting cell mixture, leaving very few islet cells in the pancreatic starting cells. In addition, our culturing techniques differ significantly with the different modes of culture, the multiple media, as well as the growth factors that are significantly different and are described below.

Another work is that of Bonner-Weir 2000 that also starts with duct enriched pancreas tissue with the statement that their approach does not actually work with the starting pancreatic cells that we are utilizing. Their culture method also relies on MATRIGEL that is not the subject of our primary approaches to permit the new cells to migrate into and form insulin-producing cells.

The third approach for developing large quantities of insulin-producing cells starts with acinar cells. Most of the early work with acinar cells was to maintain its phenotype in culture to better understand these cells (Oliver 87, Brannon 88). Then in attempting to understand the source of pancreatic cancer cells, attention turned to duct cells and the ability of acinar cells to apparently change phenotype to some sort of duct cell, as it was described. Culturing acinar cells in collagen gels, Lisle & Losdon 1990 describe the phenomenon of acinar cells losing their specific cell markers in this culture and picking up markers similar to duct cells for 6-12 days of culture, using their own monoclonal antibodies, but subsequently reverting back to their original acinar cell markers as the culture continues.

Again, interested in pancreatic cancer, Hall & Limoine 1992, describe the culture of acinar cells on plastic dishes whereby the cells began to change over 5-10 days to begin to express one of the duct cell markers CK19, but die off by 3 weeks. Arias & Bendayan 1993 cultured rat and guinea pig acinar cells on MATRIGEL with maintenance of their acinar phenotype but loss of the cells by one week. The addition of 2% DMSO to the culture of acinar cells in MATRIGEL changed the phenotype to duct-like cells that began to form cysts and tubules within the MATRIGEL. In addition, when in the cyst structures, the cells began to express CAII, a specific enzyme used by duct cells to release bicarbonate and water. Protein inhibitors prohibited the change into a duct-like phenotype. It appears that the combination of MATRIGEL and DMSO pushed the dedifferentiated islet cells on through the more primitive stage and further differentiated them into mature duct cells with a functional marker and the ability to form three dimensional structures. The question of mechanisms was raised as to whether stem cells were involved or whether this represented transdifferentiation.

Then, Bouwens 1994 studied potential duct cell markers in the neonatal rat and described that CK7 was a marker for large pancreatic ducts while CK19 was expressed in the smaller ducts, the intercalated ducts, and the centroacinar cells of the acinii. Another marker unique to the rat, CK20, marked similar cells as CK19. He also noted that while proliferation was going on, some cells next to expanding islets also expressed the CK19 or CK20. Examining mouse pancreas cells cultured on plates, Vila 1994 demonstrated human acinar cells express CK18 at the start but changed their expression to CK7 and CK19 over time with amylase levels going down. Also mucin 1 expression rose as well as another duct cell marker, CFTR, the marker for chloride transporter of duct cells. Again, the question was raised as to whether the mechanism of this change represented transdifferentiation or the involvement of stem cells. They also found that both HGF and TGFa exposure caused these cells to proliferate making the suggestion that a stem cell may be the cause and may have bearing in the development of ductal malignancies of the pancreas. But, no insulin production was observed.

Kerr-Conte 1996 demonstrated that placing purified human islets into MATRIGEL produced cystic duct-like structures that contained islet cells as small buds. It is not clear from this work as to what the source of these duct-like cells may be that could clearly proliferate, but there was no evidence of proliferation of the islet cells. Again, as previously discussed above, the suggestion that these may be dedifferentiating islet cells into duct-like cells was made, but the ability of these cells to proliferate while the differentiated cells did not proliferate raises the possibility that these cells represent stem cells. But, no insulin production was observed.

Bouwens 1998 compared the possibilities of transdifferentiation versus the role of stem cells as causing the proliferation of dedifferentiated cells from either the duct, acinar, or islet differentiated cells. While he favored the transdifferentiation mechanism due to cell markers showing the expression of the different cell types, his primary reason was because definitive stem cell markers for these cells had not yet been developed so it was not possible to specifically identify them. Yet, he acknowledged that indirect evidence can readily suggest the presence of stem cells and that the specific markers have simply not as yet been perfected. Yet again, no insulin production had been observed in his review.

Kerr-Conte 2000 and in US Patent Application (20020155598) suggests the presence of “pluripotent pancreatic stem cells” as the primary explanation of the ability to change terminally differentiated human pancreas cells to a more primitive cell type that has the ability to expand and then be differentiated into another type of specific cell that is terminally differentiated. As an accepted marker for this stem cell, she suggests the duct-like cells co-expressing CK19 and pdx1, similarly suggested by Fung, are those stem cells. She cultured a mixture of human acinar and duct cells in adherent culture showing the loss of amylase, the increase of CK19, and the increase of pdx1 expressions in the resulting duct-like cells that flattened out as monolayers. But, she was not able to show the conversion to insulin-producing cells but was able to show the new expression of a neuroendocrine cell marker, chromogranin A. In fact, her claim of pdx1 and CK19 stained cells as being evidence of precursor cells of insulin producing cells agrees with Fung and ourselves as well as with their being stem cells. But her claim that these indeed are insulin producing cells in her patent application remains unproven by her own data represented in FIGS. 4 and 5 that fails to provide any direct evidence of increased insulin production by these converted cells. Thus, she has demonstrated the presence of stem cells but fails to demonstrate their differentiation into insulin-producing cells. This is a significant difference compared to this invention where we clearly demonstrate the production of insulin-producing cells. The methods described in these two publications utilize single pancreas cells decreased in islet content, cultured in monolayers to change the acinar phenotype to the duct-like phenotype that are called ductal precursors., By her definition, these ductal precursor cells have the ability to be differentiated into insulin-producing cells. She attempts the redifferentiation by placing the ductal precursor cells into a matrix of MATRIGEL or collagen. She clearly demonstrates the ability of the ductal precursor cells to proliferate, but in the patent application, does not demonstrate the formation of any new insulin-producing cells.

There are significant differences between her techniques and those in this invention. The first step of converting the phenotype of non-insulin producing pancreatic cells to stem cells in this invention can utilize several different media in several different culture modes in addition to adherent culture using several different types of growth factors. A stem cell is formed as demonstrated by its ability to undergo replication as the intermediary, more primitive cell that carries the only makers accepted to date to identify this stem cell that are duct cell markers like CK19 and pdx1 expression in replicating cells. Her second step does not produce insulin-producing cells. In our second step, these stem cells are then differentiated into insulin producing cells by a different set of growth factors and conditions, again demonstrated in different cell culture modes. Our invention also utilizes more complex growth and differentiation factors (Table ???) than described in her publication and patent application. The normal histology and function of our new insulin-producing cells are also shown below. The definition of the stem cell used in this invention is based on the National Library of Medicine's definition that it is a cell that is not terminally differentiated that undergoes replication as well as can differentiate into more than one type of differentiated cells. Our examples show the starting non-insulin producing pancreatic cells are converted under the first set of culture conditions into stem cells that replicate and carry the CK19 and pdx1 markers. These stem cells can then be differentiated into hormone producing islet cells such as insulin or glucagon as well as into duct structures under separate differentiating conditions as described below.

Definitions:

General source of many of these definitions is OMIM, National Center for Biotechnology Information, National Library of Medicine, National Institutes of Health.

Acinar cells—pancreatic cells that make up 80% of the pancreas and produce many different enzymes including amylase, lipase, trypsin, chymotrypsin, elastase, and many others. Acinar cells can be identified by their enzyme content, by specific cytokeratins such as CK18, and by lectins against surface sialoglycoproteins. Acinar cells form spherical structural units in the pancreas called acini consisting of polarized cells that release their enzyme products into the small, centralized intercalated ducts located at the center of each acinus. Many acinar cells contain two nuclei at any time of examination of primary cells.

Duct cells—pancreatic cells making up 10% of the pancreas that define the larger interlobular and intralobular ducts as well as the smallest, intercalated ducts, that drain the pancreatic enzymes from the acini. Duct cells also produce bicarbonate and water to dilute the enzymes and alter the intestinal pH upon release into the gut from these ductal structures. Duct cells can be identified by cytokeratin subtypes such as CK19 and by the enzymes responsible for bicarbonate production.

Islet cells—endocrine cells making up 2% of the pancreas and existing as separate cell aggregates called islets that contain different cell types making different hormones. Beta cells that are 50-60% of the islet aggregate make insulin that permits glucose entry into most cells of the body. Alpha cells that are 30% of the islet make glucagon that is released during fasting to permit glucose delivery from the liver to maintain normal blood sugar. Delta cells, 10% of the islet cells, make somatostatin that fine tunes glucose levels. Pancreatic polypeptide producing cells (5-10% of the islet cells) release their hormone that alters exocrine and gastrointestinal function. In addition to these major islet cell types, there are also other islet cell types that make a variety of other hormones including GIP, VIP, gastrin, bombesin, and others. In addition, the islets contain fenestrated endothelium as a rich capillary bed into which each islet cell to releases its hormone product.

Pancreatic cells—primary pancreatic cells from human donors (or other mammalian species) that contain acinar, duct, and islet cells types as well as supportive and vascular cells.

Islet-depleted pancreatic cells—the cells remaining after the isolation of islets from a suspension of digested pancreatic cells using a discontinuous or continuous density gradient. This population is comprised mainly of acinar cells (>90%) with a small percentage of intercalated ducts within the acinar aggregates, vascular, and neuronal tissue, as well as a residual amount of contaminating islet material.

Pancreatic Acinus—any of the small spherical acinar cell structures that empty their enzyme products into the central acinar area that empties into the intercalated pancreatic ducts.

Intercalated Duct—a duct from a tubule or acinus of the pancreas that drains into an intralobular duct.

Intralobular Duct—a duct that collects pancreatic juice from the intercalated ducts and drains into an interlobular duct.

Interlobular Duct—a duct that collects pancreatic juice from intralobular ducts and drains into pancreatic ducts

Pancreatic Duct—largest of the ducts that includes the main pancreatic duct, the accessory pancreatic duct, the dorsal pancreatic duct, and the ventral pancreatic duct

Stem Cell—a cell that is not terminally differentiated that can undergo replication and can differentiate into more than one type of differentiated cell.

Cell Growth—is the replication of the cellular DNA followed by cytokinesis that can be demonstrated by BrdU or tritiated thymidine incorporation or KI67.

Cell Expansion—used to define numbers of cells that have gone through cell division and are increasing their numbers and overall mass, rather than simply enlarging by hypertrophy.

Proliferation—rapid and repeated production of new parts or of offspring (as in a mass of cells by a rapid succession of cell divisions).

Cell Hypertrophy—used to define enlarging cells that have increased their cell volume, rather than growing by cell division.

Cell Cycle—cell growth cycle. Cells that are in cell cycle have left the resting state (G₀ phase) and are replicating their contents and dividing in two.

Differentiation—is used to declare that a cell has passed from a progenitor level or more basic or generalized function to one of more specific function.

Transdifferentiation—is uses to declare that a cell has changed from a level of defined function to another.

Dedifferentiation—is used to declare that a cell has passed from a level of defined function to one of less defined function or to a basic cell.

Totipotent—capable of developing into a complete organism or differentiating into any of its cells or tissues.

Pluripotent—1: not fixed as to develop-mental potentialities: having developmental plasticity such as a pluripotent cell or pluripotent embryonic tissue. 2 capable of affecting more than one organ or tissue.

Growth Factors (GF)—include a number of compounds that may induce cell replication. There are general GF's such as Epidermal GF (EGF) and Vascular Endothelial GF (VEGF). There are also GF's that are more specific in their action. (e.g. the action of Insulin-like GF 1 (IGF1) on islets, or erythropoietin on red blood cell progenitors).

Differentiation Factors (DF)—include a number of compounds that may induce cell type specific differentiation. There are specific differentiation factors for islet cells, for acinar cells, and for duct cells. An example for acinar cells is dexamethasone.

Dedifferentiation Factors (DDF)—include a number of factors for islet cells, for acinar cells, and for duct cells that permit the cell to lose differentiated function and change to a level of function that is lower in the lineage.

Matrix or Matrices—used to define hydrogels or polymerizable materials that hold cells in place for culture under different conditions. These include MATRIGEL, collagen, alginate, and others.

Tissue Culture Flask, Dish or Plate Substrates—used to define specific types of plastic or glass surfaces that are configured either in tissue culture flasks, petri dishes or culture plates that are used to grow cells. These surfaces are prepared such that they either promote or discourage adherent or non-adherent cell growth.

Coated Culture Flask, Dish, or Plate Surfaces—a cell culture dish coated with a thin layer of a compound.

Suspension culture—cells suspended in tissue culture medium in the absence of any support from a thin layer of a compound or any matrix.

Alpha-tocopherol—any of several fat-soluble vitamins that are chemically tocopherols, are essential in the nutrition of various vertebrates in which their absence is associated with infertility, degenerative changes in muscle, or vascular abnormalities, are found especially in wheat germ, vegetable oils, egg yolk, and green leafy vegetables or are made synthetically, and are used chiefly in animal feeds and as antioxidants.

Apotransferrin—protein produced by oligodendricytes that is necessary for cell survival and involved in cell differentiation.

Atrial Natriuretic Peptide—A potent natriuretic and vasodilatory peptide or mixture of different-sized low molecular weight peptides derived from a common precursor and secreted by the heart atria. All these peptides share a sequence of about 20 amino acids.

Biotin—a colorless crystalline growth vitamin C₁₀H₁₆N₂O₃S of the vitamin B complex found especially in yeast, liver, and egg yolk.

BSA—(bovine) serum albumin is a monomeric protein that comprises about one-half of the blood's serum proteins. In vivo, it plays a role in stabilizing extracellular fluid volume and functions as a carrier for steroids, fatty acids, and some hormones.

C natriuretic peptide (CNP)—A 22-amino acid peptide that is a member of the natriuretic peptide family. It is from endothelial and renal cell origin with selective cardiovascular actions.

CAII—carbonic anhydrase type II, the enzyme used by duct cells to produce bicarbonate that is secreted into the pancreatic ducts to neutralize the acid in the duodenum generated by the stomach.

Calcium pantothenate—a white powdery salt C₁₈H₃₂C₁₈N₃₂O₁₀ made synthetically and used as a source of pantothenic acid.

Carnitine—a quaternary ammonium compound C₇H₁₅NO₃ present especially in vertebrate muscle and involved in the transfer of fatty acids across mitochondrial membranes.

Catalase—enzyme that consists of a protein complex with hematin groups and catalyzes the decomposition of hydrogen peroxide into water and oxygen

CCK—cholecystokinin is a brain and gut peptide. In the gut, it induces the release of pancreatic enzymes and the contraction of the gallbladder. It has the capacity to stimulate insulin secretion. CCK peptides exist in multiple molecular forms (e.g., sulfated CCK8, unsulfated CCK8, and CCK4), each resulting from distinct posttranslational processing of the CCK gene product.

CFTR—cystic fibrosis transmembrane conductance regulator (CFTR) functions as a chloride channel. Mutations in the CFTR gene have been found to cause cystic fibrosis. Mutations in CFTR effect the exocrine function of the pancreas, intestinal glands, biliary tree, bronchial glands and sweat glands.

CGRP alpha, (Calcitonin Gene Related Peptide)—A test that measures the amount of the hormone calcitonin in the blood.

Cholera Toxin B Subunit—The nontoxic subunit B of Cholera Toxin is important to the protein complex as it allows the protein to bind to cellular surfaces via the pentasaccharide chain of ganglioside GM 1.

CK19—cytokeratin 19 is the smallest known (40-kD) acidic keratin, one of a family of water-insoluble intermediate filaments. Different cytokeratins can be used as markers to identify certain types of epithelia and epithelial tumors. CK19 keratin is found in many types of epithelial cells, including numerous ductal and glandular epithelia. In the pancreas, it is present in ductal epithelia and absent in endocrine and exocrine cells.

CK19+cells—cytokeratin 19 is expressed in epithelial cells in culture, in particular, in “intermediary” or transdifferentiating cells from pancreatic tissues.

Corticosteroid—any of various adrenal-cortex steroids (as corticosterone, cortisone, and aldosterone) that are divided on the basis of their major biological activity into glucocorticoids and mineralocorticoids.

Corticosterone—a colorless crystalline corticosteroid C₂₁H₃₀O₄ of the adrenal cortex that is important in protein and carbohydrate metabolism.

Corticosterone (Reichstein's substance H)—a colorless crystalline corticosteroid C₂₁H₃₀O₄ of the adrenal cortex that is important in protein and carbohydrate metabolism

C-peptide—the c-peptide (“connecting” peptide) is a short polypeptide released after the conversion of proinsulin to mature insulin. Its molecular weight is 3,582 Da.

Cyclodextran—2-hyrdroypropyl-beta-cyclodextrin. A tissue culture medium additive that facilitates solubilization of hydrophobic substances.

DIF-1/Differanisole A—Differentiation-inducing factor-1 (DIF-1) is a chlorinated hexaphenone isolated from Dictyostelium. DIF-1 exhibits antitumor activity in several types of mammalian tumor cells, although the underlying mechanisms remain unknown. The structure of morphogen of Dictyostelium discoideum, DIF-1, is closely similar to that of differanisole A which had been isolated from the metabolites of a simple eukaryote, Chaetomium, as the differentiation-inducer of murine and human undifferentiated tumor cells.

DL-alpha-tocopherol acetate—a tocopherol C₂₉H₅₀O₂ with high vitamin E potency.

DMF (n-n-dimethylformamide)—affects cellular differentiation. Suppression of acidification rate is likely due to decreased metabolic acid production. Alterations in H+ production and transport contribute its effects on cellular differentiation.

DMSO—dimethyl sulfoxide (CH₃)₂SO—that is an agent known to induce cell differentiation, also a solvent, also a cryoprotectant for freezing living cells, also an anti-inflammatory agent for the treatment of interstitial cystitis

DMSO (dimethylsulfoxide)—an anti-inflammatory agent (CH₃)₂SO used in the treatment of interstitial cystitis

EGF—epidermal growth factor is a potent mitogenic factor for a variety of cultured cells of both ectodermal and mesodermal origin and has a profound effect on the differentiation of specific cells in vivo. Mature EGF is a single-chain polypeptide consisting of 53 amino acids and having a molecular mass of about 6,000.

Endothelin 1—any of several polypeptides consisting of 21 amino acid residues that are produced in various cells and tissues, that play a role in regulating vasomotor activity, cell proliferation, and the production of hormones, and that have been implicated in the development of vascular disease

Ethanolanine—a colorless liquid amino alcohol C₂H₇NO used especially as a solvent for fats and oils, —called also monoethanolamine.

Exendin 4—a long acting analog of GLP-1

FACS—fluorescence activated cell sorting

FCS—fetal calf serum. Blood serum recovered from an unborn cow.

FGF—The FGF superfamily consists of 23 known members, all of which contain a conserved 120 amino acid region. The FGFs were originally recognized to have proliferative activities; they are now considered to play substantial roles in development, angiogenesis, hematopoiesis, and tumorigenesis. Almost all of the FGFs isoforms have the ability to activate other isoform's receptors. This accounts for similar effects generated by different FGF subtypes.

FGF2—fibroblast growth factor 2 (FGF-basic) is a wide-spectrum mitogenic, angiogenic, and neurotrophic factor that is expressed at low levels in many tissues and cell types. FGF2 has been implicated in a multitude of physiologic and pathologic processes, including limb development, angiogenesis, wound healing, and tumor growth.

Galactose—an optically active sugar C₆H₁₂O₆ that is less soluble and less sweet than glucose and is known in dextrorotatory, levorotatory, and racemic forms.

GLP-1—Glucagon like-peptide 1 is a 30 amino acid peptide derived from the preproglucagon molecule. GLP-1 enhances glucose secretion and synthesis. It renders pancreatic beta-cells ‘glucose-competent’ and may be useful in the treatment of noninsulin-dependent diabetes mellitus.

GLP-2—GLP-2 is a 33-amino acid proglucagon-derived peptide. GLP-2 maintains the integrity of the intestinal mucosal epithelium via effects on gastric motility and nutrient absorption, crypt cell proliferation and apoptosis, and intestinal permeability.

Glucose—an optically active sugar C₆H₁₂O₆ that has an aldehydic carbonyl group. The breakdown of carbohydrates, particularly glucose, is a major source of energy for all plant and animal cells. In diabetes, there is a diminished ability to transport glucose into the cells of the body. Blood glucose levels are abnormally high (hyperglycemia). Elevated blood glucose can lead to ketoacidosis, resulting in coma and death. Milder hyperglycemia causes long-term complications affecting the eyes, kidneys, nerves, and blood vessels.

Glutathione—a peptide C₁₀H₁₇N₃O₆S that contains one amino acid residue each of glutamic acid, cysteine, and glycine, that occurs widely in plant and animal tissues, and that plays an important role in biological oxidation-reduction processes and as a coenzyme.

Growth hormone—growth hormone (GH) is synthesized by the anterior pituitary gland. Human growth hormone has a molecular mass of 22,005 and contains 191 amino acid residues with 2 disulfide bridges. The principal biological role of growth hormone is the control of postnatal growth. It's affect is mediated largely by insulin-like growth factors.

GRP (Gastrin Releasing Peptide)—The gastrin-releasing peptide receptor (GRP—R) can cause the proliferation of many, but not all, cells in which it is expressed.

Hb9—Homeo box-9 is one of a family of proteins that bind DNA in a sequence-specific manner and are implicated in the control of gene expression in both developing and adult tissues.

HGF—hepatocyte growth factor (also scatter factor or hepatopoietin A) has a spectrum of targets including endothelial cells and melanocytes in addition to epithelial cells such as hepatocytes. It affects diverse tissues, mediating placental growth developmental determining liver and muscle development in the embryo and B-cell proliferation and growth.

HNF3a—hepatocyte nuclear factor 3, alpha. A member of the forkhead class of transcription factors. Both HNF3A and HNF3B are expressed in tissues of endodermal origin, i.e., stomach, intestines, liver, and lung. All members of the HNF3 family as well as HNF4G and HNF6 are expressed in pancreatic beta cells

HNF6—During mouse development, Hnf6 is expressed in the epithelial cells that are precursors of the exocrine and endocrine pancreatic cells. In hnf6-null embryos, the exocrine pancreas appeared to be normal but endocrine cell differentiation was impaired. The expression of neurogenin-3, a transcription factor that is essential for determination of endocrine cell precursors, was almost abolished. Later in life, the number of endocrine cells increased but the architecture of the islets was perturbed, and the beta cells were deficient in glucose transporter-2 expression. Adult hnf6-null mice were diabetic. This suggests that Hnf6 controls embryonic pancreatic endocrine differentiation at the precursor stage and positively regulates the proendocrine gene ngn3.

HuSA—human serum albumin—see BSA (bovine serum albumin).

IBMX—3-isobutyl-1-methylxanthine A compound that inhibits cyclic AMP phosphodiesterase, which causes beta cells to release insulin.

IGF1—Insulin-like growth factor-I. Both IGF1 and IGF2 have a striking structural homology to proinsulin.

IGF2—Insulin-like growth factor 2. Both IGF1 and IGF2 have a striking structural homology to proinsulin.

Johe's N2—a serum free medium formulated for the support of multi-potential CNS stem cells is supplemented with various growth and differentiation factors

KGF—keratinocyte growth factor or FGF-7: a 28 kDa, single chain, secreted glycoprotein that has a target specificity restricted to epithelium. Adult cells known to express FGF-7 include fibroblasts, T cells, smooth muscle cells, and ovarian theca cells. In the embryo, KGF is found at many stages of development throughout the mesenchyme.

Ki67—a cell proliferation marker. This protein of unknown function is expressed during G1 of the cell cycle; it has a half-life of 60-90 minutes.

Lactogen—any hormone (as prolactin) that stimulates the production of milk.

Laminin—a glycoprotein that is a component of connective tissue basement membrane and that promotes cell adhesion.

Leu-Enkephalin—A natural peptide neurotransmitter. Natural opiate pentapeptides isolated originally from pig brain. Leu-enkephalin (YGGFL) and Met-enkephalin (YGGFM) bind particularly strongly to d-type opiate receptors.

Linoleic acid—a liquid unsaturated fatty acid C₁₈H₃₂O₂ found especially in semidrying oils (as peanut oil) and essential for the nutrition of some animals—called also linolic acid.

Linolenic acid—a liquid unsaturated fatty acid C₁₈H₃₀O₂ found especially in drying oils (as linseed oil) and essential for the nutrition of some animals.

Met-Enkephalin—A natural peptide neurotransmitter. Natural opiate pentapeptides isolated originally from pig brain. Leu-enkephalin (YGGFL) and Met-enkephalin (YGGFM) bind particularly strongly to d-type opiate receptors.

Muc 1—mucin type 1, the main type of mucoprotein normally secreted by special pancreatic duct cells.

Myoinositol—a biologically active inositol that is not optically active, that is a component of the vitamin B complex and a lipotropic agent, and that occurs widely in plants, microorganisms, and higher animals including humans—called also mesoinositol

N2—Johe's N2 medium.

Neuro—neurobasal medium, a neural cell culture medium.

NGF—Nerve growth factor is a 12.5 kDa, nonglycosylated polypeptide 120 aa residues long. It is synthesized as a prepropeptide; its processed form is a 120 aa segment. The typical form for NGF is a 25 kDa, non-disulfide linked homodimer. Nerve growth factor is known to regulate growth and differentiation of sympathetic and certain sensory neurons.

Nicotinamide—niacinamide (nicotinic acid amide) a bitter crystalline basic amide C₆H₆N₂O that is a member of the vitamin B complex and is formed from and converted to niacin in the living organism, that occurs naturally usually as a constituent of coenzymes, and that is used similarly to niacin.

PCNA+ cells—cells that label with an anti proliferating cell nuclear antigen. Proliferating cell nuclear antigen was originally correlated with the proliferative state of the cell. More recent evidence shows that PCNA may also be correlated with DNA repair.

PDGF—platelet derived growth factor. A factor released from platelets upon clotting was shown to be capable of promoting the growth of various types of cells. This factor was subsequently purified from platelets and given the name platelet-derived growth factor (PDGF). PDGF is now known to be produced by a number of cell types besides platelets and it has been found to be a mitogen for almost all mesenchymally-derived cells, i.e., blood, muscle, bone/cartilage, and connective tissue cells.

pdx-1—Pancreatic duodenal homeobox factor-1, PDX-1, is required for pancreas development, islet cell differentiation, and the maintenance of beta cell function. Also called insulin promoter factor-1 (IPF1) or IDX1 or somatostatin transcription factor-1 (STFI). PDX-1 appears to serve as a master control switch for expression of both the exocrine and endocrine pancreatic developmental programs, as revealed by gene disruption studies in which targeted deletion of the gene leads to a null pancreas phenotype. PXDX-1 is initially expressed in both exocrine and endocrine cells; as pancreatic morphogenesis proceeds, it restricted to some duct cells and beta and delta cells of the islets. PDX-1 also plays a role in adult cells, regulating the insulin and somatostatin genes. Mutations in the PDX1 gene can cause pancreatic agenesis, maturity-onset diabetes of the young, and possibly type II diabetes.

Placental lactogen—This peptide hormone is structurally, immunologically, and functionally similar to pituitary growth hormone It is synthesized by the placental syncytiotrophoblast.

Progesterone—a female steroid sex hormone C₂₁H₃₀O₂ that is secreted by the corpus luteum to prepare the endometrium for implantation and later by the placenta during pregnancy to prevent rejection of the developing embryo or fetus and that is used in synthetic forms as a birth control pill, to treat menstrual disorders, and to alleviate some cases of infertility.

Proinsulin—the precursor of insulin. Insulin is derived from a folded, one-chain precursor that is linked by 2 disulfide bonds. Proinsulin is converted to insulin by the enzymatic removal of a segment that connects the amino end of the A chain to the carboxyl end of the B chain.

Prolactin—A growth factor with strong structural similarity to growth hormone.

PTF1—see PDX-1

PTHRP—parathyroid related protein.

Putrescine—a crystalline slightly poisonous ptomaine C₄H₁₂N₂ that is formed by decarboxylation of omithine, occurs widely but in small amounts in living things, and is found especially in putrid flesh.

Reg1—regenerating-islet-derived protein Laos known as pancreatic stone protein

Retinoic Acid (Vitamin A)—a local regulator of cellular differentiation. It has many functions in the developing limb; regulates key events in limb regeneration in lower vertebrates.

Retinyl acetate—a derivative of vitamin A.

Selenium (Selenious Acid)—a nonmetallic element that resembles sulfur and tellurium chemically, causes poisoning in range animals when ingested by eating some plants growing in soils in which it occurs in quantity, and occurs in allotropic forms of which a gray stable form varies in electrical conductivity with the intensity of its illumination and is used in electronic devices.

Sonic Hedgehog (mouse, recombinant)—plays important roles in the development of many cell types including the brain, bone, skin, gonads, and lungs.

Soybean Trypsin Inhibitor (type I-S)—A high-molecular-weight protein (approximately 22,500) containing 198 amino acid residues. Soybean trypsin inhibitor suppress proteolytic but not elastolytic activity.

Substance P—Substance P is the predominant neuropeptide released at primary afferent-second order neuron synapses upon high-intensity stimulation of nociceptive afferents. Via activation of NK1 receptors (see table in chapter nociception) substance P produces slow, long-lasting depolarizations of second order neurons. This leads to potentiation of the post-synaptic response to nociceptor stimulation and thereby functions as an intensity-coding mechanism for nociceptive transmission.

Superoxide Dismutase (SOD)—a metal-containing antioxidant enzyme that reduces potentially harmful free radicals of oxygen formed during normal metabolic cell processes to oxygen and hydrogen peroxide.

TGF alpha and beta—Transforming growth factors (TGFs) are biologically active potypeptides that reversibly confer the transformed phenotype on cultured cells. Alpha-TGF shows about 40% sequence homology with epidermal growth factor. TGF beta is a multifunctional peptide that controls proliferation, differentiation, and other functions in many cell types. TGFB acts synergistically with TGFα in inducing transformation. It also acts as a negative autocrine growth factor. Dysregulation of TGFB activation and signaling may result in apoptosis. Many cells synthesize TGFB and almost all of them have specific receptors for this peptide. TGFB 1, TGFB2, and TGFB3 all function through the same receptor signaling systems.

TGF beta sR11 (soluble receptor type 2)—TGF-beta regulates growth and proliferation of cells, blocking growth of many cell types. The TGF-beta receptor includes type 1 and type 2 subunits that are serine-threonine kinases and that signal through the SMAD family of transcriptional regulators. Defects in TGF-beta signaling, includes mutation in SMADs, have been associated with cancer in humans.

Transcription Factors (TF)—Transcription factors bind to specific regulatory sequences in DNA and modulate the activity of RNA polymerase. This is the key step that regulates the process whereby genes coded in DNA are copied or transcribed into messenger RNA. Normally, the interactions of many different transcription factors determine the specific phenotype of different cell types. TF's can be positive or negative regulators of gene expression. PDX1, neurogenin 3 (ngn3), Pax4, Pax6, and others are examples of those TF's that are involved in pancreatic development and differentiation.

Transferrin—a beta globulin in blood plasma capable of combining with ferric ions and transporting iron in the body.

Triiodothyronine—a crystalline iodine-containing hormone C₁₅H₁₂I₃NO₄ that is an amino acid derived from thyroxine and is used especially in the form of its soluble sodium salt in the treatment of hypothyroidism and metabolic insufficiency—called also liothyronine, T₃.

Triiodothyronine (T3)—a crystalline iodine-containing hormone C₁₅H₁₂I₃NO₄ that is an amino acid derived from thyroxine and is used especially in the form of its soluble sodium salt in the treatment of hypothyroidism and metabolic insufficiency.

Trolox (soluble Vitamin E)—A cell-permeable, water-soluble derivative of vitamin E with potent antioxidant properties. Prevents peroxynitrite-mediated oxidative stress and apoptosis in rat thymocytes.

Vasoactive Intestinal Peptide (VIP)—A test that measures the amount of VIP in serum.

VEGF—vascular endothelial growth factor—VEGF is a heparin-binding glycoprotein that is secreted as a homodimer of 45 kDa. One of the most important growth and survival factors for endothelium. It is structurally related to platelet-derived growth factor. VEGF induces angiogenesis and endothelial cell proliferation and it plays an important role in regulating vasculogenesis. Most types of cells, but usually not endothelial cells themselves, secrete VEGF.

Zinc sulphate—Zinc is an important trace mineral and is required for the enzyme activities necessary for cell division, cell growth, and wound healing. Zinc is also involved in the metabolism of carbohydrates. Beta cells of the pancreas have a high zinc content.

### SUMMARY OF THE INVENTION

In one embodiment, the invention is drawn to a method of converting differentiated non-hormone producing pancreatic cells into differentiated hormone-producing cells, including the steps of: a) culturing the differentiated non-hormone producing pancreatic cells in a first cell culture system with a first cell culture medium including a basal medium, with or without serum, and with or without growth factors, under conditions which provide for converting the differentiated non-hormone producing pancreatic cells into stem cells; and b) culturing the stem cells in a second cell culture system with a second cell culture medium, including at least one compound-selected from Group A and at least one compound selected from Group B, where Group A includes the following compounds: Betacellulin, Activin A, BMP-2, TGF-β SR11, DMSO, Sonic Hedgehog, Laminin, Met-Enkephalin, DMF, and Cholera Toxin A; and where Group B includes the following compounds: Activin A, Atrial Natriuretic Peptide, Betacellulin, Bone Morphogenic Protein (BMP-2), Bone Morphogenic Protein (BMP-4), C natriuretic peptide (CNP), Caerulein, Calcitonin Gene Related Peptide (CGRP-α), Cholecystokinin (CCK8-amide), Cholecystokinin octapeptide (CCK8-sulfated), Cholera Toxin B Subunit, Corticosterone (Reichstein's substance H), Dexamethasone, DIF-1, Differanisole A, Dimethylsulfoxide (DMSO), EGF, Endothelin 1, Exendin 4, FGF acidic, FGF2, FGF7, FGFb, Gastrin I, Gastrin Releasing Peptide (GRP), Glucagon-Like Peptide 1 (GLP-1), Glucose, Growth Hormone, Hepatocyte Growth Factor (HGF), IGF-1, IGF-2, Insulin, KGF, Lactogen, Laminin, Leu-Enkephalin, Leukemia Inhibitory Factor (LIF), Met-Enkephalin, n Butyric Acid, Nerve Growth Factor (β-NGF), Nicotinamide, n-n-dimethylformamide (DMF), Parathyroid Hormone Related Peptide (Pth II RP), PDGF AA+PDGF BB MIX, PIGF (Placental GF), Progesterone, Prolactin, Putrescine Dihydrochloride Gamma-Irradiated Cell Culture, REG1, Retinoic Acid, Selenium, Selenious Acid, Sonic Hedgehog, Soybean Trypsin Inhibitor, Substance P, Superoxide Dismutase (SOD), TGF-α, TGF-β sRII, TGF-β1, transferrin, Triiodothyronine (T3), Trolox, Vasoactive Intestinal Peptide (VIP), VEGF, Vitamin A, and Vitamin E, under conditions which provide for differentiating the stem cells into hormone-producing cells.

In a preferred embodiment, the second cell culture medium includes at least two compounds selected from Group A and at least two compounds selected from Group B.

In a more preferred embodiment, the second cell culture medium includes at least three compounds selected from Group A and at least three compounds selected from Group B.

In a yet more preferred embodiment, the second cell culture medium includes at least four compounds selected from Group A and at least four compounds selected from Group B.

In a yet more preferred embodiment, the second cell culture medium includes at least five compounds selected from Group A and also at least five compounds selected from Group B.

In a yet more preferred embodiment, the second cell culture medium includes at least six compounds selected from Group A and at least six compounds selected from Group B.

In one embodiment, the invention is drawn to a method of culturing stem cells into differentiated hormone-producing cells, including culturing the stem cells in a cell culture system with a cell culture medium where the stem cells are differentiated into hormone-producing cells and where the culture medium includes basal medium without serum and also includes at least one compound selected from Group A and at least one compound selected from Group B, where Group A includes the following compounds: Betacellulin, Activin A, BMP-2, TGF-β SRII, DMSO, Sonic Hedgehog, Laminin, Met-Enkephalin, DMF, and Cholera Toxin A; and Group B includes the following compounds: Activin A, Atrial Natriuretic Peptide, Betacellulin, Bone Morphogenic Protein (BMP-2), Bone Morphogenic Protein (BMP-4), C natriuretic peptide (CNP), Caerulein, Calcitonin Gene Related Peptide (CGRP-α), Cholecystokinin (CCK8-amide), Cholecystokinin octapeptide (CCK8-sulfated), Cholera Toxin B Subunit, Corticosterone (Reichstein's substance H), Dexamethasone, DIF-1, Differanisole A, Dimethylsulfoxide (DMSO), EGF, Endothelin 1, Exendin 4, FGF acidic, FGF2, FGF7, FGFb, Gastrin I, Gastrin Releasing Peptide (GRP), Glucagon-Like Peptide 1 (GLP-1), Glucose, Growth Hormone, Hepatocyte Growth Factor (HGF), IGF-1, IGF-2, Insulin, KGF, Lactogen, Laminin, Leu-Enkephalin, Leukemia Inhibitory Factor (LIF), Met-Enkephalin, n Butyric Acid, Nerve Growth Factor (s-NGF), Nicotinamide, n-n-dimethylfommamide (DMF), Parathyroid Hormone Related Peptide (Pth II RP), PDGF AA+PDGF BB MIX, PIGF (Placental GF), Progesterone, Prolactin, Putrescine Dihydrochloride Gamma-Irradiated Cell Culture, REG1, Retinoic Acid, Selenium, Selenious Acid, Sonic Hedgehog, Soybean Trypsin Inhibitor, Substance P, Superoxide Dismutase (SOD), TGF-α: TGF-β sRII, TGF-β1, transferrin, Triiodothyronine (T3), Trolox, Vasoactive Intestinal Peptide (VIP), VEGF, Vitamin A, and Vitamin E.

In a preferred embodiment, the cell culture medium includes at least two compounds selected from Group A and at least two compounds selected from Group B.

In a yet more preferred embodiment, the cell culture medium includes at least three compounds selected from Group A and at least three compounds selected from Group B.

In a yet more preferred embodiment, the cell culture medium includes at least four compounds selected from Group A and at least four compounds selected from Group B.

In a yet more preferred embodiment, the cell culture medium includes at least five compounds selected from Group A and at least five compounds selected from Group B.

In a yet more preferred embodiment, the cell culture medium includes at least six compounds selected from Group A and at least six compounds selected from Group B.

### BRIEF DESCRIPTION OF THE DRAWINGS

FIG. 1. Insulin release from cells cultured in alginate in the presence of growth and differentiation factors in a combinatorial array. Donors #2212, #2278, and #3023.

FIG. 2. Stimulation index of insulin release from cells cultured in alginate in the presence of growth and differentiation factors in a combinatorial array. Donors #2212, #2278, and #3023.

FIG. 3. Insulin release from cells cultured in alginate in the top eight growth and differentiation factor combinations (A-H): Donor #2212

FIG. 4. Stimulation indices of insulin release from cells cultured in alginate in the top eight growth and differentiation factor combinations (A-H): Donor #2212.

FIG. 5. Insulin release from cells cultured in alginate in the top eight growth and differentiation factor combinations (A-H): Donor #2278

FIG. 6. Stimulation indices of insulin release from cells cultured in alginate in the top eight growth and differentiation factor combinations (A-H): Donor #2278

FIG. 7. Insulin release from cells cultured in alginate in the top eight growth and differentiation factor combinations (A-H): Donor #3023

FIG. 8. Stimulation indices of insulin release from cells cultured in alginate in the top eight growth and differentiation factor combinations (A-H): Donor #3023

FIG. 9. Insulin release from cells cultured in alginate in the top eight growth and differentiation factor combinations (A-H): Donor #3036

FIG. 10. Stimulation indices of insulin release from cells cultured in alginate in the top eight growth and differentiation factor combinations (A-H): Donor #3036

FIG. 11. Stimulation indices of c-peptide release from cells cultured in alginate in the top eight growth and differentiation-factor combinations (A-H): Donor #3036

FIG. 12. C-peptide release from cells cultured in adherent culture in the top four growth and differentiation factor combinations (I-L)

FIG. 13. Numbers of proinsulin positive cells per well of cells cultured in adherent culture in the top six growth and differentiation factor combinations determined in a second tier 30 factor screen.

### DETAILED DESCRIPTION OF THE INVENTION AND PREFERRED EMBODIMENT

In one embodiment, the invention is drawn to a method for producing a hormone producing cell from a differentiated cell type that does not produce a hormone. Preferably, the differentiated cell type is a pancreatic cell. Preferably, the cells are islet-depleted pancreatic cells. More preferably, the differentiated cell type is a non-hormone producing pancreatic cells cell.

The hormone-producing cell produced in one aspect of the present invention preferably produces one or more of the hormones produced by islet cell. More preferably, the hormone-producing cell produces insulin.

Accordingly, a preferred aspect of the invention are methods and compositions for the large scale expansion of non-hormone producing pancreatic cells and the large scale transformation of non-hormone producing pancreatic cells into hormone-producing cells. Preferably, the hormone produced is insulin but other hormones are also encompassed within the invention, particularly hormones from islet cells.

In another preferred embodiment, the invention provides compositions useful for the method of converting pancreatic non-hormone producing pancreatic cells into hormone-producing cells.

Tables 5 and 6 list factors which may be added to the culture media which include potential growth factors and potential differentiation factors. For purposes of this disclosure, the terms “factor”, “component” and “supplement” may be used interchangeably.

These components, factors and supplements include but are not limited to Activin A, Atrial Natriuretic Peptide, Betacellulin, Bone Morphogenic Protein (BMP-2), Bone Morphogenic Protein (BMP-4), C natriuretic peptide (CNP), Caerulein, Calcitonin Gene Related Peptide (CGRP-α), Cholecystokinin (CCK8-amide), Cholecystokinin octapeptide (CCK8-sulfated), Cholera Toxin B Subunit, Corticosterone (Reichstein's substance H), Dexamethasone, DIF-1, Differanisole A, Dimethylsulfoxide (DMSO), EGF, Endothelin 1, Exendin 4, FGF acidic, FGF2, FGF7, FGFb, Gastrin I, Gastrin Releasing Peptide (GRP), Glucagon-Like Peptide 1 (GLP-1), Glucose, Growth Hormone, Hepatocyte Growth Factor (HGF), IGF-1, IGF-2, Insulin, KGF, Lactogen, Laminin, Leu-Enkephalin, Leukemia Inhibitory Factor (LIF), Met-Enkephalin, n-Butyric Acid, Nerve Growth Factor (P-NGF), Nicotinamide, n-n-dimethylformamide (DMF), Parathyroid Hormone Related Peptide (Pth II RP), PDGF AA+PDGF BB MIX, PIGF (Placental GF), Progesterone, Prolactin, Putrescine Dihydrochloride Gamma-Irradiated Cell Culture, REG1, Retinoic Acid, Selenium, Selenious Acid, Sonic Hedgehog, Soybean Trypsin Inhibitor, Substance P, Superoxide Dismutase (SOD), TGF-α, TGF-β sRII, TGF-β1, transferrin, Triiodothyronine (T3), Trolox, Vasoactive Intestinal Peptide (VIP), VEGF, Vitamin A, and Vitamin E.

For Activin A, a preferred concentration is 0.125-1.5 ng/ml; yet more preferred concentration is 0.25-1 ng/ml; yet more preferred concentration is 0.375-0.75 ng/ml; yet more preferred concentration is 0.45-0.6 ng/ml; and most preferred concentration is 0.5 ng/ml.

For Atrial Natriuretic Peptide, a preferred concentration is 38.25-459 ng/ml; yet more preferred concentration is 76.5-306 ng/ml; yet more preferred concentration is 114.75-229.5 ng/ml; yet more preferred concentration is 137.7-183.6 ng/ml; and most preferred concentration is 153 ng/ml.

For Betacellulin, a preferred concentration is 1.25-15 ng/ml; yet more preferred concentration is 2.5-10 ng/ml; yet more preferred concentration is 3.75-7.5 ng/ml; yet more preferred concentration is 4.5-6 ng/ml; and most preferred concentration is 5 ng/ml.

For Bone Morphogenic Protein (BMP-2), a preferred concentration is 1.25-15 ng/ml; yet more preferred concentration is 2.5-10 ng/ml; yet more preferred concentration is 3.75-7.5 ng/ml; yet more preferred concentration is 4.5-6 ng/ml; and most preferred concentration is 5 ng/ml.

For Bone Morphogenic Protein (BMP-4), a preferred concentration is 0.125-1.5 ng/ml; yet more preferred concentration is 0.25-1 ng/ml; yet more preferred concentration is 0.375°-0.75 ng/ml; yet more preferred concentration is 0.45°-0.6 ng/ml; and most preferred concentration is 0.5 ng/ml.

For C natriuretic peptide (CNP), a preferred concentration is 27.4625-329.55 ng/ml; yet more preferred concentration is 54.925-219.7 ng/ml; yet more preferred concentration is 82.3875-164.775 ng/ml; yet more preferred concentration is 98.865-131.82 ng/ml; and most preferred concentration is 109.85 ng/ml.

For Caerulein, a preferred concentration is 7.5-90 ng/ml; yet more preferred concentration is 15-60 ng/ml; yet more preferred concentration is 22.5-45 ng/ml; yet more preferred concentration is 27-36 ng/ml; and most preferred concentration is 30 ng/ml.

For Calcitonin Gene Related Peptide (CGRP—), a preferred concentration is 47.625-571.5 ng/ml; yet more preferred concentration is 95.25-381 ng/ml; yet more preferred concentration is 142.875-285.75 ng/ml; yet more preferred concentration is 171.45-228.6 ng/ml; and most preferred concentration is 190.5 ng/ml.

For Cholecystokinin (CCK8-amide), a preferred concentration is 6.25-75 ng/ml; yet more preferred concentration is 12.5-50 ng/ml; yet more preferred concentration is 18.75-37.5 ng/ml; yet more preferred concentration is 22.5-30 ng/ml; and most preferred concentration is 25 ng/ml.

For Cholecystokinin octapeptide (CCK8-sulfated), a preferred concentration is 1.425-17.1 ng/ml; yet more preferred concentration is 2.85-11.4 ng/ml; yet more preferred concentration is 4.275-8.55 ng/ml; yet more preferred concentration is 5.13-6.84 ng/ml; and most preferred concentration is 5.7 ng/ml.

For Cholecystokinin octapeptide (CCK8-sulfated), a preferred concentration is 3.125-37.5 ng/ml; yet more preferred concentration is 6.25-25 ng/ml; yet more preferred concentration is 9.375-18.75 ng/ml; yet more preferred concentration is 11.25-15 ng/ml; and most preferred concentration is 12.5 ng/ml.

For Corticosterone (Reichstein's substance H), a preferred concentration is 0.5-6 ng/ml; yet more preferred concentration is 1-4 ng/ml; yet more preferred concentration is 1.5-3 ng/ml; yet more preferred concentration is. 1.8-2.4 ng/ml; and most preferred concentration is 2 ng/ml.

For Dexamethasone, a preferred concentration is 0.5-6 ng/ml; yet more preferred concentration is 1-4 ng/ml; yet more preferred concentration is 1.5-3 ng/ml; yet more preferred concentration is 1.8-2.4 ng/ml; and most preferred concentration is 2 ng/ml.

For DIF-1, a preferred concentration is 75-900 ng/ml; yet more preferred concentration is 150-600 ng/ml; yet more preferred concentration is 225-450 ng/ml; yet more preferred concentration is 270-360 ng/ml; and most preferred concentration is 300 ng/ml.

For Differanisole A, a preferred concentration is 75-900 ng/ml; yet more preferred concentration is 150-600 ng/ml; yet more preferred concentration is 225-450 ng/ml; yet more preferred concentration is 270-360 ng/ml; and most preferred concentration is 300 ng/ml.

For Dimethylsulfoxide (DMSO), a preferred concentration is 0.25-3 ng/ml; yet more preferred concentration is 0.5-2 ng/ml; yet more preferred concentration is 0.75-1.5 ng/ml; yet more preferred concentration is 0.9-1.2 ng/ml; and most preferred concentration is 1 ng/ml.

For EGF, a preferred concentration is 1.25-15 ng/ml; yet more preferred concentration is 2.5-10 ng/ml; yet more preferred concentration is 3.75-7.5 ng/ml; yet more preferred concentration is 4.5-6 ng/ml; and most preferred concentration is 5 ng/ml.

For Endothelin 1, a preferred concentration is 125-1500 ng/ml; yet more preferred concentration is 250-1000 ng/ml; yet more preferred concentration is 375-750 ng/ml; yet more preferred concentration is 450-600 ng/ml; and most preferred concentration is 500 ng/ml.

For Exendin 4, a preferred concentration is 5.25-63 ng/ml; yet more preferred concentration is 10.5-42 ng/ml; yet more preferred concentration is 15.75-31.5 ng/ml; yet more preferred concentration is 18.9-25.2 ng/ml; and most preferred concentration is 21 ng/ml.

For FGF acidic, a preferred concentration is 0.625-7.5 ng/ml; yet more preferred concentration is 1.25-5 ng/ml; yet more preferred concentration is 1.875-3.75 ng/ml; yet more preferred concentration is 2.25-3 ng/ml; and most preferred concentration is 2.5 ng/ml.

For FGF2, a preferred concentration is 0.625-7.5 ng/ml; yet more preferred concentration is 1.25-5 ng/ml; yet more preferred concentration is 1.875-3.75 ng/ml; yet more preferred concentration is 2.25-3 ng/ml; and most preferred concentration is 2.5 ng/ml.

For FGF7, a preferred concentration is 0.625-7.5 ng/ml; yet more preferred concentration is 1.25-5 ng/ml; yet more preferred concentration is 1.875-3.75 ng/ml; yet more preferred concentration is 2.25-3 ng/ml; and most preferred concentration is 2.5 ng/ml.

For FGFb, a preferred concentration is 0.625-7.5 ng/ml; yet more preferred concentration is 1.25-5 ng/ml; yet more preferred concentration is 1.875-3.75 ng/ml; yet more preferred concentration is 2.25-3 ng/ml; and most preferred concentration is 2.5 ng/ml.

For Gastrin I, a preferred concentration is 0.008038-0.09645 ng/ml; yet more preferred concentration is 0.016075-0.0643 ng/ml; yet more preferred concentration is 0.024113-0.048225 ng/ml; yet more preferred concentration is 0.028935-0.03858 ng/ml; and most preferred concentration is 0.03215 ng/ml.

For Gastrin Releasing Peptide (GRP), a preferred concentration is 35.75-429 ng/ml; yet more preferred concentration is 71.5-286 ng/ml; yet more preferred concentration is 107.25-214.5 ng/ml; yet more preferred concentration is 128.7-171.6 ng/ml; and most preferred concentration is 143 ng/ml.

For Glucagon-Like Peptide 1 (GLP-1), a preferred concentration is 8.25-99 ng/ml; yet more preferred concentration is 16.5-66 ng/ml; yet more preferred concentration is 24.75-49.5 ng/ml; yet more preferred concentration is 29.7-39.6 ng/ml; and most preferred concentration is 33 ng/ml.

For Glucose, a preferred concentration is 270-3240 ng/ml; yet more preferred concentration is 540-2160 ng/ml; yet more preferred concentration is 810-1620 ng/ml; yet more preferred concentration is 972-1296 ng/ml; and most preferred concentration is 1080 ng/ml.

For Growth Hormone, a preferred concentration is 6.25-75 ng/ml; yet more preferred concentration is 12.5-50 ng/ml; yet more preferred concentration is 18.75-37.5 ng/ml; yet more preferred concentration is 22.5-30 ng/ml; and most preferred concentration is 25 ng/ml.

For Hepatocyte Growth Factor (HGF), a preferred concentration is 0.625-7.5 ng/ml; yet more preferred concentration is 1.25-5 ng/ml; yet more preferred concentration is 1.875-3.75 ng/ml; yet more preferred concentration is 2.25-3 ng/ml; and most preferred concentration is 2.5 ng/ml.

For IGF-1, a preferred concentration is 0.625-7.5 ng/ml; yet more preferred concentration is 1.25-5 ng/ml; yet more preferred concentration is 1.875-3.75 ng/ml; yet more preferred concentration is 2.25-3 ng/ml; and most preferred concentration is 2.5 ng/ml.

For IGF-2, a preferred concentration is 0.625-7.5 ng/ml; yet more preferred concentration is 1.25-5 ng/ml; yet more preferred concentration is 1.875-3.75 ng/ml; yet more preferred concentration is 2.25-3 ng/ml; and most preferred concentration is 2.5 ng/ml.

For Insulin, a preferred concentration is 2375-28500 ng/ml; yet more preferred concentration is 4750-19000 ng/ml; yet more preferred concentration is 7125-14250 ng/ml; yet more preferred concentration is 8550-11400 ng/ml; and most preferred concentration is 9500 ng/ml.

For KGF, a preferred concentration is 0.625-7.5 ng/ml; yet more preferred concentration is 1.25-5 ng/ml; yet more preferred concentration is 1.875-3.75 ng/ml; yet more preferred concentration is 2.25-3 ng/ml; and most preferred concentration is 2.5 ng/ml.

For Lactogen, a preferred concentration is 12.5-150 ng/ml; yet more preferred concentration is 25-100 ng/ml; yet more preferred concentration is 37.5-75 ng/ml; yet more preferred concentration is 45-60 ng/ml; and most preferred concentration is 50 ng/ml.

For Laminin, a preferred concentration is 562.5-6750 ng/ml; yet more preferred concentration is 1-125-4500 ng/ml; yet more preferred concentration is 1687.5-3375 ng/ml; yet more preferred concentration is 2025-2700 ng/ml; and most preferred concentration is 2250 ng/ml.

For Leu-Enkephalin, a preferred concentration is 0.75-9 ng/ml; yet more preferred concentration is 1.5-6 ng/ml; yet more preferred concentration is 2.25-4.5 ng/ml; yet more preferred concentration is 2.7-3.6 ng/ml; and most preferred concentration is 3 ng/ml.

For Leukemia Inhibitory Factor (LIF), a preferred concentration is 0.625-7.5 ng/ml; yet more preferred concentration is 1.25-5 ng/ml; yet more preferred concentration is 1.875-3.75 ng/ml; yet more preferred concentration is 2.25-3 ng/ml; and most preferred concentration is 2.5 ng/ml.

For Met-Enkephalin, a preferred concentration is 0.75-9 ng/ml; yet more preferred concentration is 1.5-6 ng/ml; yet more preferred concentration is 2.25-4.5 ng/ml; yet more preferred concentration is 2.7-3.6 ng/ml; and most preferred concentration is 3 ng/ml.

For n-Butyric Acid, a preferred concentration is 1135-13620 ng/ml; yet more preferred concentration is 2270-9080 ng/ml; yet more preferred concentration is 3405-6810 ng/ml; yet more preferred concentration is 4086-5448 ng/ml; and most preferred concentration is 4540 ng/ml.

For Nerve Growth Factor (—NGF), a preferred concentration is 0.625-7.5 ng/ml; yet more preferred concentration is 1.25-5; ng/ml; yet more preferred concentration is 1.875-3.75 ng/ml; yet more preferred concentration is 2.25-3 ng/ml; and most preferred concentration is 2.5 ng/ml.

For Nicotinamide, a preferred concentration is 152500-1830000 ng/ml; yet more preferred concentration is 305000-1220000 ng/ml; yet more preferred concentration is 457500-915000 ng/ml; yet more preferred concentration is 549000-732000 ng/ml; and most preferred concentration is 610000 ng/ml.

For n-n-dimethylformamide (DMF), a preferred concentration is 0.25-3×10⁻⁶ percent; yet more preferred concentration is 0.5-2×10⁻⁶ percent; yet more preferred concentration is 0.75-1.5×10⁻⁶ percent; yet more preferred concentration is 0.9-1.2×10-6 percent; and most preferred concentration is 1×10⁻⁶ percent.

For Parathyroid Hormone Related Peptide —(Pth II RP), a preferred concentration is 51.5-618 ng/ml; yet more preferred concentration is 103-412 ng/ml; yet more preferred concentration is 154.5-309 ng/ml; yet more preferred concentration is 185.4-247.2 ng/ml; and most preferred concentration is 206 ng/ml.

For PDGF AA+PDGF BB mix, a preferred concentration is 1.25-15 ng/ml; yet more preferred concentration is 2.5-10 ng/ml; yet more preferred concentration is 3.75-7.5 ng/ml; yet more preferred concentration is 4.5-6 ng/ml; and most preferred concentration is 5 ng/ml.

For PIGF (Placental GF), a preferred concentration is 0.625-7.5 ng/ml; yet more preferred concentration is 1.25-5 ng/ml; yet more preferred concentration is 1.875-3.75 ng/ml; yet more preferred concentration is 2.25-3 ng/ml; and most preferred concentration is 2.5 ng/ml.

For Progesterone, a preferred concentration is 0.75-9 ng/ml; yet more preferred concentration is 1.5-6 ng/ml; yet more preferred concentration is 2.25-4.5 ng/ml; yet more preferred concentration is 2.7-3.6 ng/ml; and most preferred concentration is 3 ng/ml.

For Prolactin, a preferred concentration is 0.3-3.6 ng/ml; yet more preferred concentration is 0.6-2.4 ng/ml; yet more preferred concentration is 0.9-1.8 ng/ml; yet more preferred concentration is 1.08-1.44 ng/ml; and most preferred concentration is 1.2 ng/ml.

For Putrescine Dihydrochloride Gamma-Irradiated Cell Culture, a preferred concentration is 0.025-0.3 ng/ml; yet more preferred concentration is 0.05-0.2 ng/ml; yet more preferred concentration is 0.075-0.15 ng/ml; yet more preferred concentration is 0.09-0.12 ng/ml; and most preferred concentration is 0.1 ng/ml.

For REG1, a preferred concentration is 8.1375-97.65 ng/ml; yet more preferred concentration is 16.275-65.1 ng/ml; yet more preferred concentration is 24.4125-48.825 ng/ml; yet more preferred concentration is 29.295-39.06 ng/ml; and most preferred concentration is 32.55 ng/ml.

For Retinoic Acid, a preferred concentration is 6.25-75 ng/ml; yet more preferred concentration is 12.5-50 ng/ml; yet more preferred concentration is 18.75-37.5 ng/ml; yet more preferred concentration is 22.5-30 ng/ml; and most preferred concentration is 25 ng/ml.

For Selenium (Selenious Acid), a preferred concentration is 6.25-75 ng/ml; yet more preferred concentration is 12.5-50 ng/ml; yet more preferred concentration is 18.75-37.5 ng/ml; yet more preferred concentration is 22.5-30 ng/ml; and most preferred concentration is 25 ng/ml.

For Sonic Hedgehog, a preferred concentration is 6.25-75 ng/ml; yet more preferred concentration is 12.5-50 ng/ml; yet more preferred concentration is 18.75-37.5 ng/ml; yet more preferred concentration is 22.5-30 ng/ml; and most preferred concentration is 25 ng/ml.

For Soybean Trypsin Inhibitor, a preferred concentration is 250-3000 ng/ml; yet more preferred concentration is 500-2000 ng/ml; yet more preferred concentration is 750-1500 ng/ml; yet more preferred concentration is 900-1200 ng/ml; and most preferred concentration is 1000 ng/ml.

For Substance P, a preferred concentration is 1250-15000 ng/ml; yet more preferred concentration is 2500-10000 ng/ml; yet more preferred concentration is 3750-7500 ng/ml; yet more preferred concentration is 4500-6000 ng/ml; and most preferred concentration is 5000 ng/ml.

For Superoxide Dismutase (SOD), a preferred concentration is 2.5-30 IU/ml; yet more preferred concentration is 5-20 IU/ml; yet more preferred concentration is 7.5-15 IU/ml; yet more preferred concentration is 9-12 IU/ml; and most preferred concentration is 10 IU/ml.

For TGF-α, a preferred concentration is 0.25-3 ng/ml; yet more preferred concentration is 0.5-2 ng/ml; yet more preferred concentration is 0.75-1.5 ng/ml; yet more preferred concentration is 0.9-1.2 ng/ml and most preferred concentration is 1 ng/ml.

For TGF-β sRII, a preferred concentration is 1.25-15 ng/ml; yet more preferred concentration is 2.5-10 ng/ml; yet more preferred concentration is 3.75-7.5 ng/ml; yet more preferred concentration is 4.5-6 ng/ml; and most preferred concentration is 5 ng/ml.

For TGF-β1, a preferred concentration is 0.125-1.5 ng/ml; yet more preferred concentration is 0.25-1 ng/ml; yet more preferred concentration is 0.375-0.75 ng/ml; yet more preferred concentration is 0.45-0.6 ng/ml; and most preferred concentration is 0.5 ng/ml.

For transferrin, a preferred concentration is 687.5-8250 ng/ml; yet more preferred concentration is 1375-5500 ng/ml; yet more preferred concentration is 2062.5-4125 ng/ml; yet more preferred concentration is 2475-3300 ng/ml; and most preferred concentration is 2750 ng/ml.

For Triiodothyronine (T3), a preferred concentration is 8.375-100.5 ng/ml; yet more preferred concentration is 16.75-67 ng/ml; yet more preferred concentration is 25.125-50.25 ng/ml; yet more preferred concentration is 30.15-40.2 ng/ml; and most preferred concentration is 33.5 ng/ml.

For Trolox, a preferred concentration is 156.25-1875 ng/ml; yet more preferred concentration is 312.5-1250 ng/ml; yet more preferred concentration is 468.75-937.5 ng/ml; yet more preferred concentration is 562.5-750 ng/ml; and most preferred concentration is 625 ng/ml.

For Vasoactive Intestinal Peptide (VIP), a preferred concentration is 16.625-199.5 ng/ml; yet more preferred concentration is 33.25-133 ng/ml; yet more preferred concentration is 49.875-99.75 ng/ml; yet more preferred concentration is 59.85-79.8 ng/ml; and most preferred concentration is 66.5 ng/ml.

For VEGF, a preferred concentration is 0.625-7.5 ng/ml; yet more preferred concentration is 1.25-5 ng/ml; yet more preferred concentration is 1.875-3.75 ng/ml; yet more preferred concentration is 2.25-3 ng/ml; and most preferred concentration is 2.5 ng/ml.

For Vitamin A, a preferred concentration is 6.25-75 ng/ml; yet more preferred concentration is 12.5-50 ng/ml; yet more preferred concentration is 18.75-37.5 ng/ml; yet more preferred concentration is 22.5-30 ng/ml; and most preferred concentration is 25 ng/ml.

For soluble Vitamin E, a preferred concentration is 156.25-1875 ng/ml; yet more preferred concentration is 312.5-1250 ng/ml; yet more preferred concentration is 468.75-937.5 ng/ml; yet more preferred concentration is 562.5-750 ng/ml; and most preferred concentration is 625 ng/ml.

In one embodiment, stem cells are cultured with a mode of suspension, adherent or matrix in a cell culture medium, with or without serum, containing compounds listed in any column of Table 1. More preferably, the culture mode is MATRIGEL, collagen, hydrogel, or other crosslinkable gel matrix. More preferably, the culture mode is a hydrogel matrix. Most preferably, the culture mode is an alginate matrix.

In one embodiment, stem cells are cultured in a cell culture medium, with or without serum, containing compounds listed in Column A, Table 1. Preferably, the culture medium contains at least one of the factors and supplements listed in Column A, Table 1. More preferably, the culture medium contains at least two of the factors and supplements listed in Column A, Table 1. More preferably, the culture medium contains at least three of the factors and supplements listed in Column A, Table 1. More preferably, the culture medium contains at least four of the factors and supplements listed in Column A, Table 1. More preferably, the culture medium contains at least five of the factors and supplements listed in Column A, Table 1. More preferably, the culture medium contains at least six of the factors and supplements listed in Column A, Table 1. More preferably, the culture medium contains at least seven of the factors and supplements listed in Column A, Table 1. More preferably, the culture medium contains at least eight of the factors and supplements listed in Column A, Table 1. More preferably, the culture medium contains at least nine of the factors and supplements listed in Column A, Table 1. More preferably, the culture medium contains at least ten of the factors and supplements listed in Column A, Table 1. More preferably, the culture medium contains at least 11 of the factors and supplements listed in Column A, Table 1. More preferably, the culture medium contains at least 12 of the factors and supplements listed in Column A, Table 1. More preferably, the culture medium contains at least 13 of the factors and supplements listed in Column A, Table 1. More preferably, the culture medium contains at least 14 of the factors and supplements listed in Column A, Table 1. More preferably, the culture medium contains at least 15 of the factors and supplements listed in Column A, Table 1. More preferably, the culture medium contains at least 16 of the factors and supplements listed in Column A, Table 1. More preferably, the culture medium contains at least 17 of the factors and supplements listed in Column A, Table 1. More preferably, the culture medium contains at least 18 of the factors and supplements listed in Column A, Table 1. More preferably, the culture medium contains at least 19 of the factors and supplements listed in Column A, Table 1. More preferably, the culture medium contains at least 20 of the factors and supplements listed in Column A, Table 1. More preferably, the culture medium contains at least 21 of the factors and supplements listed in Column A, Table 1. More preferably, the culture medium contains at least 22 of the factors and supplements listed in Column A, Table 1. More preferably, the culture medium contains at least 23 of the factors and supplements listed in Column A, Table 1. More preferably, the culture medium contains at least 24 of the factors and supplements listed in Column A, Table 1. More preferably, the culture medium contains at least 25 of the factors and supplements listed in Column A, Table 1. More preferably, the culture medium contains at least 26 of the factors and supplements listed in Column A, Table 1. More preferably, the culture medium contains at least 27 of the factors and supplements listed in Column A, Table 1. More preferably, the culture medium contains at least 28 of the factors and supplements listed in Column A, Table 1. Most preferably, the culture medium contains all the factors and supplements listed in Column A, Table 1.

In one embodiment, stem cells are cultured in a cell culture medium, with or without serum, containing compounds listed in Column B, Table 1. Preferably, the culture medium contains at least one of the factors and supplements listed in Column B, Table 1. More preferably, the culture medium contains at least two of the factors and supplements listed in Column B, Table 1. More preferably, the culture medium contains at least three of the factors and supplements listed in Column B, Table 1. More preferably, the culture medium contains at least four of the factors and supplements listed in Column B, Table 1. More preferably, the culture medium contains at least five of the factors and supplements listed in Column B, Table 1. More preferably, the culture medium contains at least six of the factors and supplements listed in Column B, Table 1. More preferably, the culture medium contains at least seven of the factors and supplements listed in Column B, Table 1. More preferably, the culture medium contains at least eight of the factors and supplements listed in Column B, Table 1. More preferably, the culture medium contains at least nine of the factors and supplements listed in Column B, Table 1. More preferably, the culture medium contains at least ten of the factors and supplements listed in Column B, Table 1. More preferably, the culture medium contains at least 11 of the factors and supplements listed in Column B, Table 1. More preferably, the culture medium contains at least 12 of the factors and supplements listed in Column B, Table 1. More preferably, the culture medium contains at least 13 of the factors and supplements listed in Column B, Table 1. More preferably, the culture medium contains at least 14 of the factors and supplements listed in Column B, Table 1. More preferably, the culture medium contains at least 15 of the factors and supplements listed in Column B, Table 1. More preferably, the culture medium contains at least 16 of the factors and supplements listed in Column B, Table 1. More preferably, the culture medium contains at least 17 of the factors and supplements listed in Column B, Table 1. More preferably, the culture medium contains at least 18 of the factors and supplements listed in Column B, Table 1. More preferably, the culture medium contains at least 19 of the factors and supplements listed in Column B, Table 1. More preferably, the culture medium contains at least 20 of the factors and supplements listed in Column B, Table 1. More preferably, the culture medium contains at least 21 of the factors and supplements listed in Column B, Table 1. More preferably, the culture medium contains at least 22 of the factors and supplements listed in Column B, Table 1. More preferably, the culture medium contains at least 23 of the factors and supplements listed in Column B, Table 1. More preferably, the culture medium contains at least 24 of the factors and supplements listed in Column B, Table 1. More preferably, the culture medium contains at least 25 of the factors and supplements listed in Column B, Table 1. More preferably, the culture medium contains at least 26 of the factors and supplements listed in Column B, Table 1. More preferably, the culture medium contains at least 27 of the factors and supplements listed in Column B, Table 1. More preferably, the culture medium contains at least 28 of the factors and supplements listed in Column B, Table 1. More preferably, the culture medium contains at least 29 of the factors and supplements listed in Column B, Table 1. More preferably, the culture medium contains at least 30 of the factors and supplements listed in Column B, Table 1. More preferably, the culture medium contains at least 31 of the factors and supplements listed in Column B, Table 1. Most preferably, the culture medium contains all the factors and supplements listed in Column B, Table 1.

In one embodiment, stem cells are cultured in a cell culture medium, with or without serum, containing compounds listed in Column C, Table 1. Preferably, the culture medium contains at least one of the factors and supplements listed in Column C, Table 1. More preferably, the culture medium contains at least two of the factors and supplements listed in Column C, Table 1. More preferably, the culture medium contains at least three of the factors and supplements listed in Column C, Table 1. More preferably, the culture medium contains at least four of the factors and supplements listed in Column C, Table 1. More preferably, the culture medium contains at least five of the factors and supplements listed in Column C, Table 1. More preferably, the culture medium contains at least six of the factors and supplements listed in Column C, Table 1. More preferably, the culture medium contains at least seven of the factors and supplements listed in Column C, Table 1. More preferably, the culture medium contains at least eight of the factors and supplements listed in Column C, Table 1. More preferably, the culture medium contains at least nine of the factors and supplements listed in Column C, Table 1. More preferably, the culture medium contains at least ten of the factors and supplements listed in Column C, Table 1. More preferably, the culture medium contains at least 11 of the factors and supplements listed in Column C, Table 1. More preferably, the culture medium contains at least 12 of the factors and supplements listed in Column C, Table 1. More preferably, the culture medium contains at least 13 of the factors and supplements listed in Column C, Table 1. More preferably, the culture medium contains at least 14 of the factors and supplements listed in Column C, Table 1. More preferably, the culture medium contains at least 15 of the factors and supplements listed in Column C, Table 1. More preferably; the culture medium contains at least 16 of the factors and supplements listed in Column C, Table 1. More preferably, the culture medium contains at least 17 of the factors and supplements listed in Column C, Table 1. More preferably, the culture medium contains at least 18 of the factors and supplements listed in Column C, Table 1. More preferably, the culture medium contains at least 19 of the factors and supplements listed in Column C, Table 1. More preferably, the culture medium contains at least 20 of the factors and supplements listed in Column C, Table 1. More preferably, the culture medium contains at least 21 of the factors and supplements listed in Column C, Table 1. More preferably, the culture medium contains at least 22 of the factors and supplements listed in Column C, Table 1. More preferably, the culture medium contains at least 23 of the factors and supplements listed in Column C, Table 1. More preferably, the culture medium contains at least 24 of the factors and supplements listed in Column C, Table 1. More preferably, the culture medium contains at least 25 of the factors and supplements listed in Column C, Table 1. More preferably, the culture medium contains at least 26 of the factors and supplements listed in Column C, Table 1. More preferably, the culture medium contains at least 27 of the factors and supplements listed in Column C, Table 1. Most preferably, the culture medium contains all the factors and supplements listed in Column C, Table 1.

In one embodiment, stem cells are cultured in a cell culture medium, with or without serum, containing compounds listed in Column D, Table 1. Preferably, the culture medium contains at least one of the factors and supplements listed in Column D, Table 1. More preferably, the culture medium contains at least two of the factors and supplements listed in Column D, Table 1. More preferably, the culture medium contains at least three of the factors and supplements listed in Column D, Table 1. More preferably, the culture medium contains at least four of the factors and supplements listed in Column D, Table 1. More preferably, the culture medium contains at least five of the factors and supplements listed in Column D, Table 1. More preferably, the culture medium contains at least six of the factors and supplements listed in Column D, Table 1. More preferably, the culture medium contains at least seven of the factors and supplements listed in Column D, Table 1. More preferably, the culture medium contains at least eight of the factors and supplements listed in Column D, Table 1. More preferably, the culture medium contains at least nine of the factors and supplements listed in Column D, Table 1. More preferably, the culture medium contains at least ten of the factors and supplements listed in Column D, Table 1. More preferably, the culture medium contains at least 11 of the factors and supplements listed in Column D, Table 1. More preferably, the culture medium contains at least 12 of the factors and supplements listed in Column D, Table 1. More preferably, the culture medium contains at least 13 of the factors and supplements listed in Column D, Table 1. More preferably, the culture medium contains at least 14 of the factors and supplements listed in Column D, Table 1. More preferably, the culture medium contains at least 15 of the factors and supplements listed in Column D, Table 1. More preferably, the culture medium contains at least 16 of the factors and supplements listed in Column D, Table 1. More preferably, the culture medium contains at least 17 of the factors and supplements listed in Column D, Table 1. More preferably, the culture medium contains at least 18 of the factors and supplements listed in Column D, Table 1. More preferably, the culture medium contains at least 19 of the factors and supplements listed in Column D, Table 1. More preferably, the culture medium contains at least 20 of the factors and supplements listed in Column D, Table 1. More preferably, the culture medium contains at least 21 of the factors and supplements listed in Column D, Table 1. More preferably, the culture medium contains at least 22 of the factors and supplements listed in Column D, Table 1. More preferably, the culture medium contains at least 23 of the factors and supplements listed in Column D, Table 1. Most preferably, the culture medium contains all the factors and supplements listed in Column D, Table 1.

In one embodiment, stem cells are cultured in a cell culture medium, with or without serum, containing compounds listed in Column E, Table 1. Preferably, the culture medium contains at least one of the factors and supplements listed in Column E, Table 1. More preferably, the culture medium contains at least two of the factors and supplements listed in Column E, Table 1. More preferably, the culture medium contains at least three of the factors and supplements listed in Column E, Table 1. More preferably, the culture medium contains at least four of the factors and supplements listed in Column E, Table 1. More preferably, the culture medium contains at least five of the factors and supplements listed in Column E, Table 1. More preferably, the culture medium contains at least six of the factors and supplements listed in Column E, Table 1. More preferably, the culture medium contains at least seven of the factors and supplements listed in Column E, Table 1. More preferably, the culture medium contains at least eight of the factors and supplements listed in Column E, Table 1. More preferably, the culture medium contains at least nine of the factors and supplements listed in Column E, Table 1. More preferably, the culture medium contains at least ten of the factors and supplements listed in Column E, Table 1. More preferably, the culture medium contains at least 11 of the factors and supplements listed in Column E, Table 1. More preferably, the culture medium contains at least 12 of the factors and supplements listed in Column E, Table 1. More preferably, the culture medium contains at least 13 of the factors and supplements listed in Column E, Table 1. More preferably, the culture medium contains at least 14 of the factors and supplements listed in Column E, Table 1. More preferably, the culture medium contains at least 15 of the factors and supplements listed in Column E, Table 1. More preferably, the culture medium contains at least 16 of the factors and supplements listed in Column E, Table 1. More preferably, the culture medium contains at least 17 of the factors and supplements listed in Column E, Table 1. More preferably, the culture medium contains at least 18 of the factors and supplements listed in Column E, Table 1. More preferably, the culture medium contains at least 19 of the factors and supplements listed in Column E, Table 1. More preferably, the culture medium contains at least 20 of the factors and supplements listed in Column E, Table 1. More preferably, the culture medium contains at least 21 of the factors and supplements listed in Column E, Table 1. More preferably, the culture medium contains at least 22 of the factors and supplements listed in Column E, Table 1. More preferably, the culture medium contains at least 23 of the factors and supplements listed in Column E, Table 1. More preferably, the culture medium contains at least 24 of the factors and supplements listed in Column E, Table 1. More preferably, the culture medium contains at least 25 of the factors and supplements listed in Column E, Table 1. More preferably, the culture medium contains at least 26 of the factors and supplements listed in Column E, Table 1. More preferably, the culture medium contains at least 27 of the factors and supplements listed in Column E, Table 1. More preferably, the culture medium contains at least 28 of the factors and supplements listed in Column E, Table 1. More preferably, the culture medium contains at least 29 of the factors and supplements listed in Column E, Table 1. More preferably, the culture medium contains at least 30 of the factors and supplements listed in Column E, Table 1. More preferably, the culture medium contains at least 31 of the factors and supplements listed in Column E, Table 1. More preferably, the culture medium contains at least 32 of the factors and supplements listed in Column E, Table 1. Most preferably, the culture medium contains all the factors and supplements listed in Column E, Table 1.

In one embodiment, stem cells are cultured in a cell culture medium, with or without serum, containing compounds listed in Column F, Table 1. Preferably, the culture medium contains at least one of the factors and supplements listed in Column F, Table 1. More preferably, the culture medium contains at least two of the factors and supplements listed in Column F, Table 1. More preferably, the culture medium contains at least three of the factors and supplements listed in Column F, Table 1. More preferably, the culture medium contains at least four of the factors and supplements listed in Column F, Table 1. More preferably, the culture medium contains at least five of the factors and supplements listed in Column F, Table 1. More preferably, the culture medium contains at least six of the factors and supplements listed in Column F, Table 1. More preferably, the culture medium contains at least seven of the factors and supplements listed in Column F, Table 1. More preferably, the culture medium contains at least eight of the factors and supplements listed in Column F, Table 1. More preferably, the culture medium contains at least nine of the factors and supplements listed in Column F, Table 1. More preferably, the culture medium contains at least ten of the factors and supplements listed in Column F, Table 1. More preferably, the culture medium contains at least 11 of the factors and supplements listed in Column F, Table 1. More preferably, the culture medium contains at least 12 of the factors and supplements listed in Column F, Table 1. More preferably, the culture medium contains at least 13 of the factors and supplements listed in Column F, Table 1. More preferably, the culture medium contains at least 14 of the factors and supplements listed in Column F, Table 1. More preferably, the culture medium contains at least 15 of the factors and supplements listed in Column F, Table 1. More preferably, the culture medium contains at least 16 of the factors and supplements listed in Column F, Table 1. More preferably, the culture medium contains at least. 17 of the factors and supplements listed in Column F, Table 1. More preferably; the culture medium contains at least 18 of the factors and supplements listed in Column F, Table 1. More preferably, the culture medium contains at least 19 of the factors and supplements listed in Column F, Table 1. More preferably, the culture medium contains at least 20 of the factors and supplements listed in Column F, Table 1. More preferably, the culture medium contains at least 21 of the factors and supplements listed in Column F, Table 1. More preferably, the culture medium contains at least 22 of the factors and supplements listed in Column F, Table 1. More preferably, the culture medium contains at least 23 of the factors and supplements listed in Column F, Table 1. More preferably, the culture medium contains at least 24 of the factors and supplements listed in Column F, Table 1. More preferably, the culture medium contains at least 25 of the factors and supplements listed in Column F, Table 1. More preferably, the culture medium-contains at least 26 of the factors and supplements listed in Column F, Table 1. More preferably, the culture medium contains at least 27 of the factors and supplements listed in Column F, Table 1. More preferably, the culture medium contains at least 28 of the factors and supplements listed in Column F, Table 1. More preferably, the culture medium contains at least 29 of the factors and supplements listed in Column F, Table 1. More preferably, the culture medium contains at least 30 of the factors and supplements listed in Column F, Table 1. More preferably, the culture medium contains at least 31 of the factors and supplements listed in Column F, Table 1. More preferably, the culture medium contains at least 32 of the factors and supplements listed in Column F, Table 1. Most preferably, the culture medium contains all the factors and supplements listed in Column F, Table 1.

In one embodiment, stem cells are cultured in a cell culture medium, with or without serum, containing compounds listed in Column G, Table 1. Preferably, the culture medium contains at least one of the factors and supplements listed in Column G, Table 1. More preferably, the culture medium contains at least two of the factors and supplements listed in Column G, Table 1. More preferably, the culture medium contains at least three of the factors and supplements listed in Column G, Table 1. More preferably, the culture medium contains at least four of the factors and supplements listed in Column G, Table 1. More preferably, the culture medium contains at least five of the factors and supplements listed in Column G, Table 1. More preferably, the culture medium contains at least six of the factors and supplements listed in Column G, Table 1. More preferably, the culture medium contains at least seven of the factors and supplements listed in Column G, Table 1. More preferably, the culture medium contains at least eight of the factors and supplements listed in Column G, Table 1. More preferably, the culture medium contains at least nine of the factors and supplements listed in Column G, Table 1. More preferably; the culture medium contains at least ten of the factors and supplements listed in Column G, Table 1. More preferably, the culture medium contains at least 11 of the factors and supplements listed in Column G, Table 1. More preferably, the culture medium contains at least 12 of the factors and supplements listed in Column G, Table 1. More preferably, the culture medium contains at least 13 of the factors and supplements listed in Column G, Table 1. More preferably, the culture medium contains at least 14 of the factors and supplements listed in Column G, Table 1. More preferably, the culture medium contains at least 15 of the factors and supplements listed in Column G, Table 1. More preferably, the culture medium contains at least 16 of the factors and supplements listed in Column G, Table 1. More preferably, the culture medium contains at least 17 of the factors and supplements listed in Column G, Table 1. More preferably, the culture medium contains at least 18 of the factors and supplements listed in Column G, Table 1. More preferably, the culture medium contains at least 19 of the factors and supplements listed in Column G, Table 1. More preferably, the culture medium contains at least 20 of the factors and supplements listed in Column G. Table 1. More preferably, the culture medium contains at least 21 of the factors and supplements listed in Column G, Table 1. More preferably, the culture medium contains at least 22 of the factors and supplements listed in Column G, Table 1. More preferably, the culture medium contains at least 23 of the factors and supplements listed in Column G, Table 1. More preferably, the culture medium contains at least 24 of the factors and supplements listed in Column G, Table 1. More preferably, the culture medium contains at least 25 of the factors and supplements listed in Column G, Table 1. More preferably, the culture medium contains at least 26 of the factors and supplements listed in Column G, Table 1. More preferably, the culture medium contains at least 27 of the factors and supplements listed in Column G, Table 1. More preferably, the culture medium contains at least 28 of the factors and supplements listed in Column G, Table 1. More preferably, the culture medium contains at least 29 of the factors and supplements listed in Column G, Table 1. Most preferably, the culture medium contains all the factors and supplements listed in Column G, Table 1.

In one embodiment, stem cells are cultured in a cell culture medium, with or without serum, containing compounds listed in Column H, Table 1. Preferably, the culture medium contains at least one of the factors and supplements listed in Column H, Table 1. More preferably, the culture medium contains at least two of the factors and supplements listed in Column H, Table 1. More preferably, the culture medium contains at least three of the factors and supplements listed in Column H, Table 1. More preferably, the culture medium contains at least four of the factors and supplements listed in Column H, Table 1. More preferably, the culture medium contains at least five of the factors and supplements listed in Column H, Table 1. More preferably, the culture medium contains at least six of the factors and supplements listed in Column H, Table 1. More preferably, the culture medium contains at least seven of the factors and supplements listed in Column H, Table 1. More preferably, the culture medium contains at least eight of the factors and supplements listed in Column H, Table 1. More preferably, the culture medium contains at least nine of the factors and supplements listed in Column H, Table 1. More preferably, the culture medium contains at least ten of the factors and supplements listed in Column H, Table 1. More preferably, the culture medium contains at least 11 of the factors and supplements listed in Column H, Table 1. More preferably, the culture medium contains at least 12 of the factors and supplements listed in Column H, Table 1. More preferably, the culture medium contains at least 13 of the factors and supplements listed in Column H, Table 1. More preferably, the culture medium contains at least 14 of the factors and supplements listed in Column H, Table 1. More preferably, the culture medium contains at least 15 of the factors and supplements listed in Column H, Table 1. More preferably, the culture medium contains at least 16 of the factors and supplements listed in Column H, Table 1. More preferably, the culture medium contains at least 17 of the factors and supplements listed in Column H, Table 1. More preferably, the culture medium contains at least 18 of the factors and supplements listed in Column H, Table 1. More preferably, the culture medium contains at least 19 of the factors and supplements listed in Column H, Table 1. More preferably, the culture medium contains at least 20 of the factors and supplements listed in Column H, Table-1. More preferably, the culture medium contains at least 21 of the factors and supplements listed in Column H, Table 1. More preferably, the culture medium contains at least 22 of the factors and supplements listed in Column H, Table 1. More preferably, the culture medium contains at least 23 of the factors and supplements listed in Column H, Table 1. More preferably, the culture medium contains at least 24 of the factors and supplements listed in Column H, Table 1. More preferably, the culture medium contains at least 25 of the factors and supplements listed in Column H, Table 1. More preferably, the culture medium contains at least 26 of the factors and supplements listed in Column H, Table 1. More preferably, the culture medium contains at least 27 of the factors and supplements listed in Column H, Table 1. More preferably, the culture medium contains at least 28 of the factors and supplements listed in Column H, Table 1. More preferably, the culture medium contains at least 29 of the factors and supplements listed in Column H, Table 1. More preferably, the culture medium contains at least 30 of the factors and supplements listed in Column H, Table 1. More preferably, the culture medium contains at least 31 of the factors and supplements listed in Column H, Table 1. More preferably, the culture medium contains at least 32 of the factors and supplements listed in Column H, Table 1. More preferably, the culture medium contains at least 33 of the factors and supplements listed in Column H, Table 1. Most preferably, the culture medium contains all the factors and supplements listed in Column H, Table 1.

In one embodiment, stem cells are cultured with a mode of suspension, adherent or matrix in a cell culture medium, with or without serum, containing compounds listed in any column of Table 2. More preferably, the culture mode is adherent. Most preferably, the culture mode is an alginate adherent.

In one embodiment, stem cells are cultured in a cell culture medium, with or without serum, containing compounds listed in Column I, Table 2. Preferably, the culture medium contains at least one of the factors and supplements listed in Column I, Table 2. More preferably, the culture medium contains at least two of the factors and supplements listed in Column I, Table 2. More preferably, the culture medium contains at least three of the factors and supplements listed in Column I, Table 2. More preferably, the culture medium contains at least four of the factors and supplements listed in Column I, Table 2. More preferably, the culture medium contains at least five of the factors and supplements listed in Column I, Table 2. More preferably, the culture medium contains at least six of the factors and supplements listed in Column I, Table 2. More preferably, the culture medium contains at least seven of the factors and supplements listed in Column I, Table 2. More preferably, the culture medium contains at least eight of the factors and supplements listed in Column I, Table 2. More preferably, the culture medium contains at least nine of the factors and supplements listed in Column I, Table 2. More preferably, the culture medium contains at least ten of the factors and supplements listed in Column I, Table 2. More preferably, the culture medium contains at least 11 of the factors and supplements listed in Column I, Table 2. More preferably, the culture medium contains at least 12 of the factors and supplements listed in Column I, Table 2. More preferably, the culture medium contains at least 13 of the factors and supplements listed in Column I, Table 2. More preferably, the culture medium contains at least 14 of the factors and supplements listed in Column I, Table 2. More preferably, the culture medium contains at least 15 of the factors and supplements listed in Column I, Table 2. More preferably, the culture medium contains at least 16 of the factors and supplements listed in Column I, Table 2. More preferably, the culture medium contains at least 17 of the factors and supplements listed in Column I, Table 2. More preferably, the culture medium contains at least 18 of the factors and supplements listed in Column I, Table 2. More preferably, the culture medium contains at least 19 of the factors and supplements listed in Column I, Table 2. More preferably, the culture medium contains at least 20 of the factors and supplements listed in Column I, Table 2. More preferably, the culture medium contains at least 21 of the factors and supplements listed in Column I, Table 2. More preferably, the culture medium contains at least 22 of the factors and supplements listed in Column I, Table 2. More preferably, the culture medium contains at least 23 of the factors and supplements listed in Column I, Table 2. More preferably, the culture medium contains at least 24 of the factors and supplements listed in Column I, Table 2. More preferably, the culture medium contains at least 25 of the factors and supplements listed in Column I, Table 2. More preferably, the culture medium contains at least 26 of the factors and supplements listed in Column I, Table 2. More preferably, the culture medium contains at least 27 of the factors and supplements listed in Column I, Table 2. More preferably, the culture medium contains at least 28 of the factors and supplements listed in Column I, Table 2. More preferably, the culture medium contains at least 29 of the factors and supplements listed in Column I, Table 2. More preferably, the culture medium contains at least 30 of the factors and supplements listed in Column I, Table 2. More preferably, the culture medium contains at least 31 of the factors and supplements listed in Column I, Table 2. More preferably, the culture medium contains at least 32 of the factors and supplements listed in Column I, Table 2. Most preferably, the culture medium contains all the factors and supplements listed in Column I, Table 2.

In one embodiment, stem cells are cultured in a cell culture medium, with or without serum, containing compounds listed in Column J, Table 2. Preferably, the culture medium contains at least one of the factors and supplements listed in Column J, Table 2. More preferably, the culture medium contains at least two of the factors and supplements listed in Column J, Table 2. More preferably, the culture medium contains at least three of the factors and supplements listed in Column J, Table 2. More preferably, the culture medium contains at least four of the factors and supplements listed in Column J, Table 2. More preferably, the culture medium contains at least five of the factors and supplements listed in Column J, Table 2. More preferably, the culture medium contains at least six of the factors and supplements listed in Column J, Table 2. More preferably, the culture medium contains at least seven of the factors and supplements listed in Column J, Table 2. More preferably, the culture medium contains at least eight of the factors and supplements listed in Column J, Table 2. More preferably, the culture medium contains at least nine of the factors and supplements listed in Column J, Table 2. More preferably, the culture medium contains at least ten of the factors and supplements listed in Column J, Table 2. More preferably, the culture medium contains at least 11 of the factors and supplements listed in Column J, Table 2. More preferably, the culture medium contains at least 12 of the factors and supplements listed in Column J, Table 2. More preferably, the culture medium contains at least 13 of the factors and supplements listed in Column J, Table 2. More preferably, the culture medium contains at least 14 of the factors and supplements listed in Column J, Table 2. More preferably, the culture medium contains at least 15 of the factors and supplements listed in Column J, Table 2. More preferably, the culture medium contains at least 16 of the factors and supplements listed in Column J, Table 2. More preferably, the culture medium contains at least 17 of the factors and supplements listed in Column J, Table 2. More preferably, the culture medium contains at least 18 of the factors and supplements listed in Column J, Table 2. More preferably, the culture medium contains at least 19 of the factors and supplements listed in Column J, Table 2. More preferably, the culture medium contains at least 20 of the factors and supplements listed in Column J, Table 2. More preferably, the culture medium contains at least 21 of the factors and supplements listed in Column J, Table 2. More preferably, the culture medium contains at least 22 of the factors and supplements listed in Column J, Table 2. More preferably, the culture medium contains at least 23 of the factors and supplements listed in Column J, Table 2. More preferably, the culture medium contains at least 24 of the factors and supplements listed in Column J, Table 2. More preferably, the culture medium contains at least 25 of the factors and supplements listed in Column J, Table 2. More preferably, the culture medium contains at least 26 of the factors and supplements listed in Column J, Table 2. More preferably, the culture medium contains at least 27 of the factors and supplements listed in Column J, Table 2. More preferably, the culture medium contains at least 28 of the factors and supplements listed in Column J, Table 2. More preferably, the culture medium contains at least 29 of the factors and supplements listed in Column J, Table 2. More preferably, the culture medium contains at least 30 of the factors and supplements listed in Column J, Table 2. More preferably, the culture medium contains at least 31 of the factors and supplements listed in Column J, Table 2. Most preferably, the culture medium contains all the factors and supplements listed in Column J, Table 2.

In one embodiment, stem cells are cultured in a cell culture medium, with or without serum, containing compounds listed in Column K, Table 2. Preferably, the culture medium contains at least one of the factors and supplements listed in Column K, Table 2. More preferably, the culture medium contains at least two of the factors and supplements listed in Column K, Table 2. More preferably, the culture medium contains at least three of the factors and supplements listed in Column K, Table 2. More preferably, the culture medium contains at least four of the factors and supplements listed in Column K, Table 2. More preferably, the culture medium contains at least five of the factors and supplements listed in Column K, Table 2. More preferably, the culture medium contains at least six of the factors and supplements listed in Column K, Table 2. More preferably, the culture medium contains at least seven of the factors and supplements listed in Column K, Table 2. More preferably, the culture medium contains at least eight of the factors and supplements listed in Column K, Table 2. More preferably, the culture medium contains at least nine of the factors and supplements listed in Column K, Table 2. More preferably, the culture medium contains at least ten of the factors and supplements listed in Column K, Table 2. More preferably, the culture medium contains at least 11 of the factors and supplements listed in Column K, Table 2. More preferably, the culture medium contains at least 12 of the factors and supplements listed in Column K, Table 2. More preferably, the culture medium contains at least 13 of the factors and supplements listed in Column K, Table 2. More preferably, the culture medium contains at least 14 of the factors and supplements listed in Column K, Table 2. More preferably, the culture medium contains at least 15 of the factors and supplements listed in Column K, Table 2. More preferably, the culture medium contains at least 16 of the factors and supplements listed in Column K, Table 2. More preferably, the culture medium contains at least 17 of the factors and supplements listed in Column K, Table 2. More preferably, the culture medium contains at least 18 of the factors and supplements listed in Column K, Table 2. More preferably, the culture medium contains at least 19 of the factors and supplements listed in Column K, Table 2. More preferably, the culture medium contains at least 20 of the factors and supplements listed in Column K, Table 2. More preferably, the culture medium contains at least 21 of the factors and supplements listed in Column K, Table 2. More preferably, the culture medium contains at least 22 of the factors and supplements listed in Column K, Table 2. More preferably, the culture medium contains at least 23 of the factors and supplements listed in Column K, Table 2. More preferably, the culture medium contains at least 24 of the factors and supplements listed in Column K, Table 2. More preferably, the culture medium contains at least 25 of the factors and supplements listed in Column K, Table 2. More preferably, the culture medium contains at least 26 of the factors and supplements listed in Column K, Table 2. More preferably, the culture medium contains at least 27 of the factors and supplements listed in Column K, Table 2. More preferably, the culture medium contains at least 28 of the factors and supplements listed in Column K, Table 2. More preferably, the culture medium contains at least 29 of the factors and supplements listed in Column K, Table 2. More preferably, the culture medium contains at least 30 of the factors and supplements listed in Column K, Table 2. Most preferably, the culture medium contains all the factors and supplements listed in Column K, Table 2.

In one embodiment, stem cells are cultured in a cell culture medium, with or without serum, containing compounds listed in Column L, Table 2. Preferably, the culture medium contains at least one of the factors and supplements listed in Column L, Table 2. More preferably, the culture medium contains at least two of the factors and supplements listed in Column L, Table 2. More preferably, the culture medium contains at least three of the factors and supplements listed in Column L, Table 2. More preferably, the culture medium contains at least four of the factors and supplements listed in Column L, Table 2. More preferably, the culture medium contains at least five of the factors and supplements listed in Column L, Table 2. More preferably, the culture medium contains at least six of the factors and supplements listed in Column L, Table 2. More preferably, the culture medium contains at least seven of the factors and supplements listed in Column L, Table 2. More preferably, the culture medium contains at least eight of the factors and supplements listed in Column L, Table 2. More preferably, the culture medium contains at least nine of the factors and supplements listed in Column L, Table 2. More preferably, the culture medium contains at least ten of the factors and supplements listed in Column L, Table 2. More preferably, the culture medium contains at least 11 of the factors and supplements listed in Column L, Table 2. More preferably, the culture medium contains at least 12 of the factors and supplements listed in Column L, Table 2. More preferably, the culture medium contains at least 13 of the factors and supplements listed in Column L, Table 2. More preferably, the culture medium contains at least 14 of the factors and supplements listed in Column L, Table 2. More preferably, the culture medium contains at least 15 of the factors and supplements listed in Column L, Table 2. More preferably, the culture medium contains at least 16 of the factors and supplements listed in Column L, Table 2. More preferably, the culture medium contains at least 17 of the factors and supplements listed in Column L, Table 2. More preferably, the culture medium contains at least 18 of the factors and supplements listed in Column L, Table 2. More preferably, the culture medium contains at least 19 of the factors and supplements listed in Column L, Table 2. More preferably, the culture medium contains at least 20 of the factors and supplements listed in Column L, Table 2. More preferably, the culture medium contains at least 21 of the factors and supplements listed in Column L, Table 2. More preferably, the culture medium contains at least 22 of the factors and supplements listed in Column L, Table 2. More preferably, the culture medium contains at least 23 of the factors and supplements listed in Column L, Table 2. More preferably, the culture medium contains at least 24 of the factors and supplements listed in Column L, Table 2. More preferably, the culture medium contains at least 25 of the factors and supplements listed in Column L, Table 2. More preferably, the culture medium contains at least 26 of the factors and supplements listed in Column L, Table 2. More preferably, the culture medium contains at least 27 of the factors and supplements listed in Column L, Table 2. More preferably, the culture medium contains at least 28 of the factors and supplements listed in Column L, Table 2. More preferably, the culture medium contains at least 29 of the factors and supplements listed in Column L, Table 2. Most preferably, the culture medium contains all the factors and supplements listed in Column L, Table 2.

### EXAMPLES

### Example 1

Sequential Culture of Pancreatic Cells in Alginate Followed by Suspension Culture

Pancreatic cells were cultured for 6-12 days in 1.6% alginate in a medium consisting of a mixture of DMEM and Ham's F12 nutrient mixture supplemented with 10% FBS, insulin, transferrin, selenium and EGF resulting in the generation of stem cells. Stem cells were harvested from the alginate beads by depolymerization and cultured in suspension in ultra low adherence plates (Costar) for 11 days in basal medium supplemented with combinations of 60 growth and differentiation factors in a 120 combinatorial array. At the end of the culture period cells were subjected to a 24 hr challenge with basal glucose medium (5 mM glucose), 20 mM glucose or 20 mM glucose+IBMX. Supernatants were harvested and analyzed for insulin content using an ELISA. Cells were washed and lysed and the DNA content per well determined using a picogreen assay The “insulin difference” was calculated by the subtraction of the insulin content in wells stimulated with basal medium from the insulin content in the supernatants in wells after stimulation with glucose alone or in combination with IBMX. Insulin difference of supernatants generated after stimulation with glucose alone ranged from 0.007-0.9908 ng/well and from 0.0098-1.1523 ng/well after stimulation with glucose and IBMX. Many wells produced low levels of insulin as calculated by the insulin difference. A few wells produced significant amounts of insulin compared to control wells assayed prior to the addition of factors in the combinatorial array as well as control wells cultured in basal medium without additional growth and differentiation factors.

These data show that selecting specific growth and differentiation factors, in combination, can be used with different culture modes in order to promote the differentiation of a pancreatic stem cell into an insulin producing cell.

### Example 2

Sequential culture of stem cells in alginate followed by adherent culture.

Pancreatic cells were cultured for 6-12 days in 1.6% alginate in a medium consisting of a mixture of DMEM and Ham's F12 nutrient mixture supplemented with 10% FBS, insulin, transferrin, selenium and EGF resulting in the generation of stem cells. Stem cells were harvested from the alginate beads by depolymerization, and cultured in adherent culture, on collagen coated plates for 8 days in basal medium supplemented with combinations of 60 growth factors in a 120 combinatorial array. At the end of the culture period cells were subjected to a 24 hr challenge with basal glucose medium (5 mM glucose), 20 mM glucose or 20 mM glucose+1 mM IBMX. Supernatants were harvested and analyzed for insulin content using an ELISA. Cells were washed and lysed and the DNA content per well determined using a picogreen assay. The “insulin difference” was calculated by the subtraction of the insulin content in wells stimulated with basal medium from the insulin content in the supernatants in wells after stimulation with glucose alone or in combination with IBMX. Insulin difference of supernatants generated after stimulation with glucose alone ranged from 0.0019-0.97-14 ng/well and from 0.0052-0.9524 ng/well after stimulation with glucose and IBMX. Many wells produced low levels of insulin as calculated by the insulin difference. A few wells produced significant amounts of insulin compared to control wells assayed prior to the addition of factors in the combinatorial array as well as control wells cultured in basal medium without additional growth and differentiation factors.

These data show that selecting specific growth and differentiation factors, in combination, can be used with different culture modes in order to promote the differentiation of a pancreatic stem cell into an insulin producing cell.

### Example 3

Culture of Stem Cells in Alginate Culture.

Pancreatic cells were cultured for 6-12 days in 1.6% alginate in a medium consisting of a mixture of DMEM and Ham's F12 nutrient mixture supplemented with 10% FBS, insulin, transferrin, selenium and EGF resulting in the generation of stem cells. Stem cells were harvested from the alginate beads by depolymerization, and recast into 1.2% alginate beads and cultured for an additional 7-11 days in basal medium supplemented with combinations of 60 growth factors in a 120 combinatorial array. At the end of the culture period cells were subjected to a 24 hr challenge with basal glucose medium (5 mM glucose), 20 mM glucose or 20 mM glucose+1 mM IBMX. Supernatants were harvested and analyzed for insulin and C-peptide content using an ELISA. Alginate beads were depolymerized and the cells were washed and lysed and the DNA content per well determined using a picogreen assay

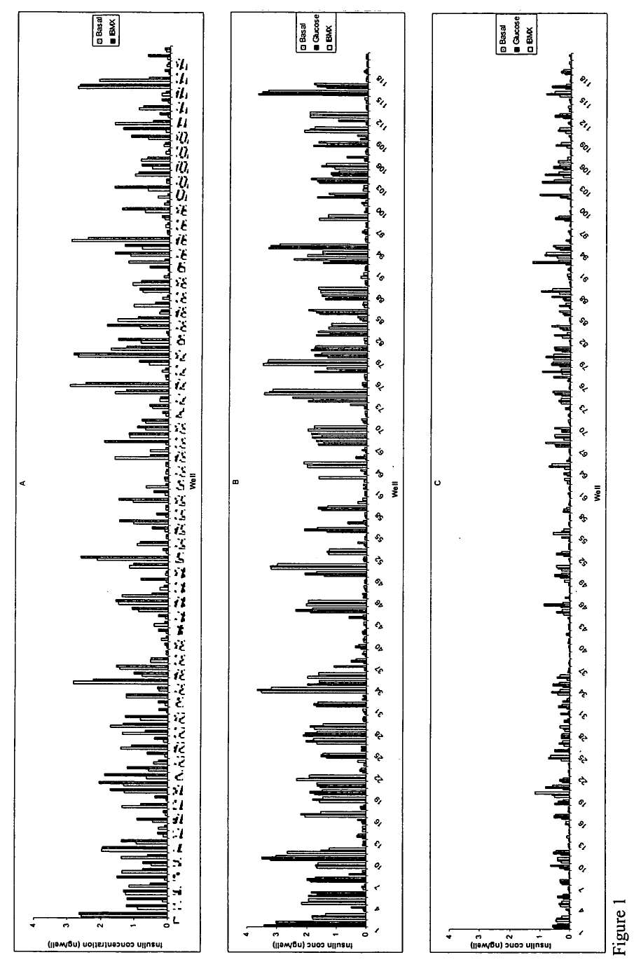

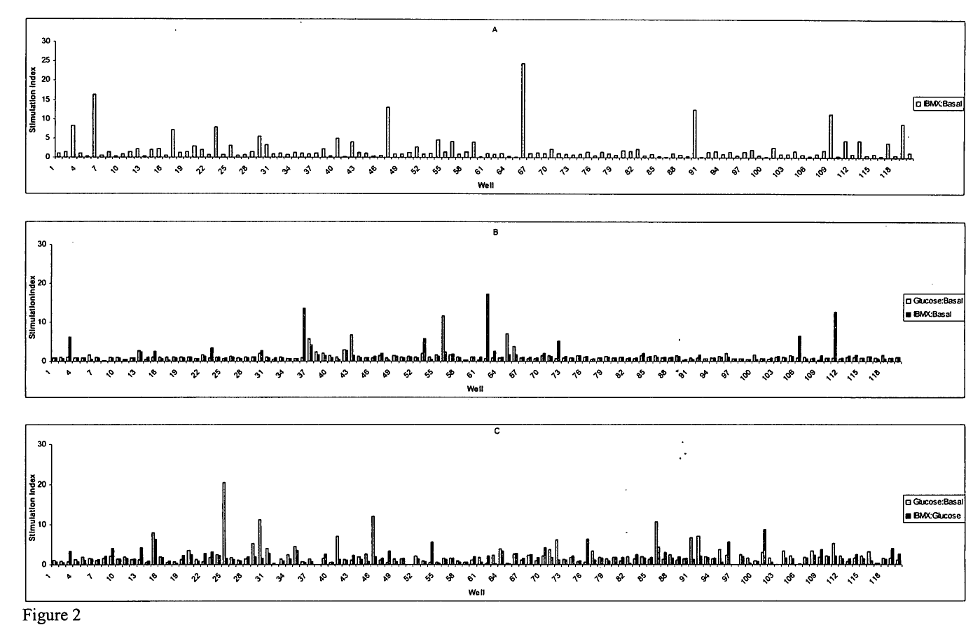

Insulin and c-peptide data from 4 replicate experiments using material from four separate human donors were examined with results examined from duplicate wells. Wells that showed consistent stimulation of insulin release were identified by comparison of the level of insulin or -peptide induced by incubation in the presence of glucose or glucose and IBMX to that produced by wells incubated in basal medium. Insulin assays were performed on all wells to determine which well combinations of growth and differentiation factors produced significant stimulated insulin release: The results of these assays are plotted in FIG. 1. These plots show insulin content following either basal glucose, high glucose or high glucose plus IBMX for each well in the combinatorial array. Many of these show wells with very little insulin, some wells show high basal levels of insulin as well as high stimulation and others with significant stimulated release. To aid in the picking of the best well combinations, a stimulation index was calculated for the high glucose divided by the basal or IBMX divided by the basal. These results are shown in FIG. 2. These clearly show several candidates for best wells using these results. Several additional analyses were done to determine which were the eight best wells that were selected.

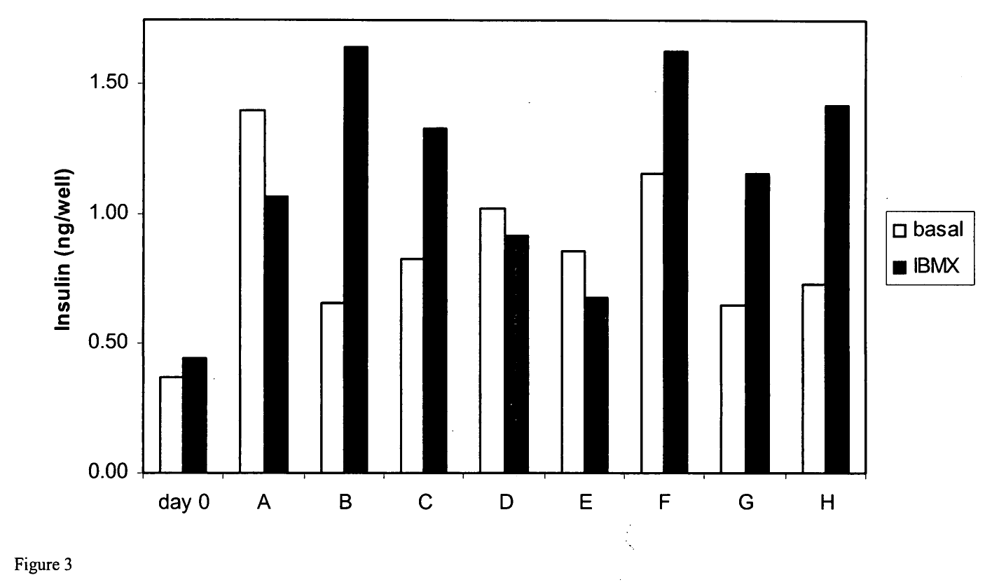

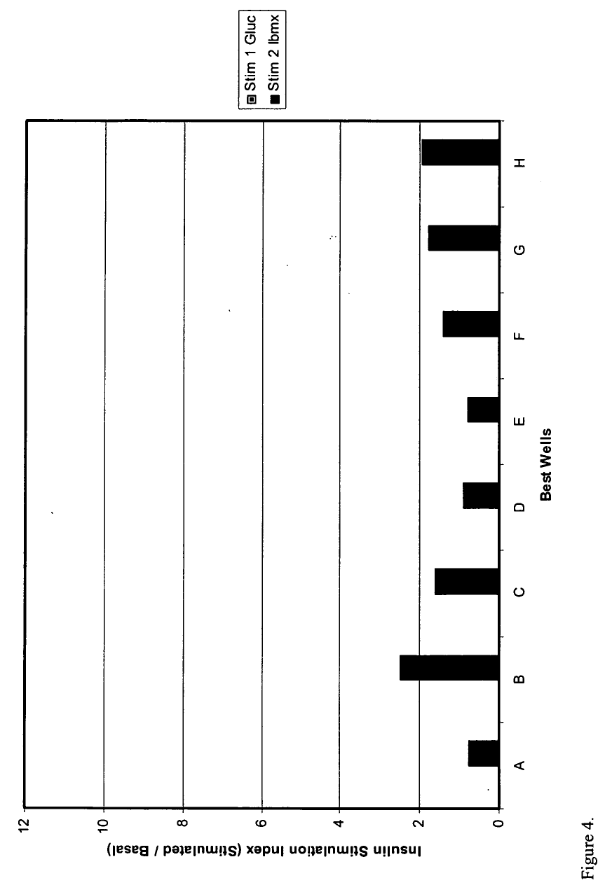

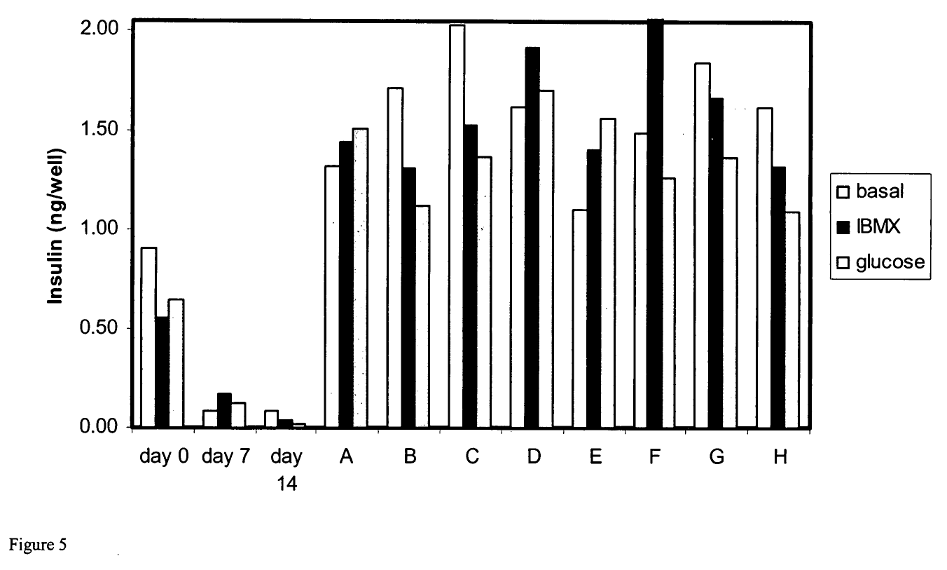

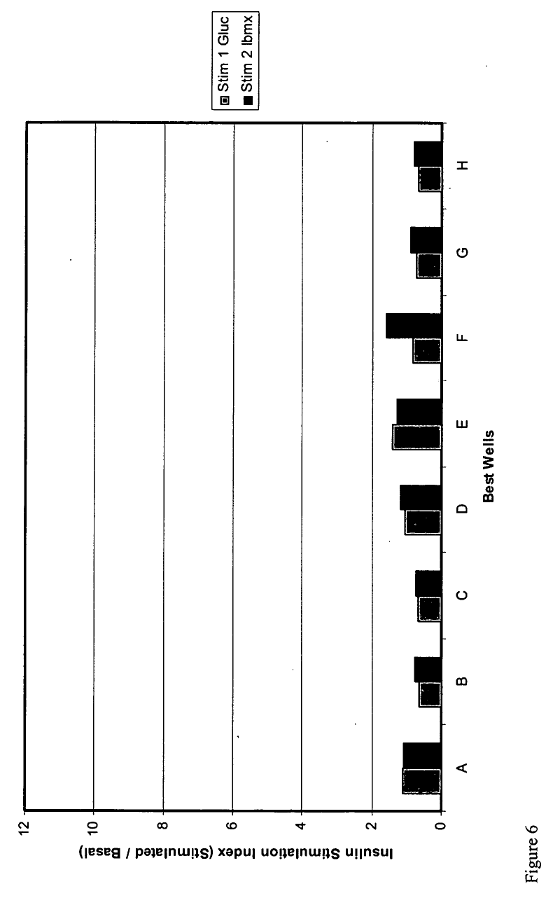

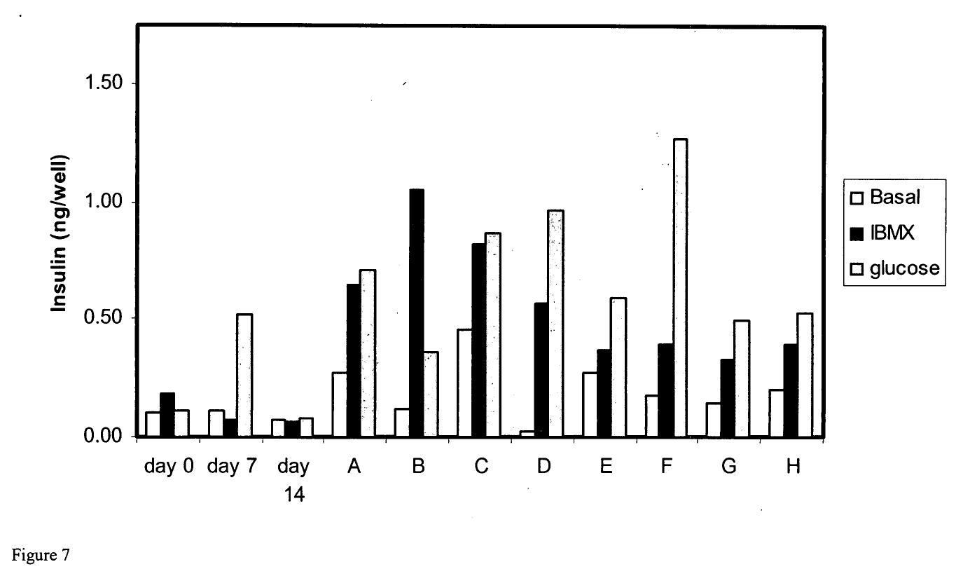

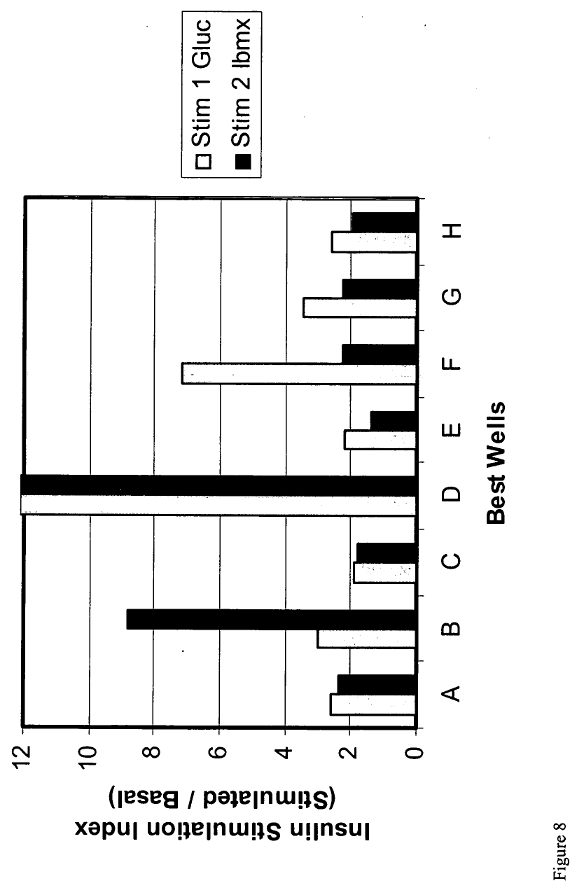

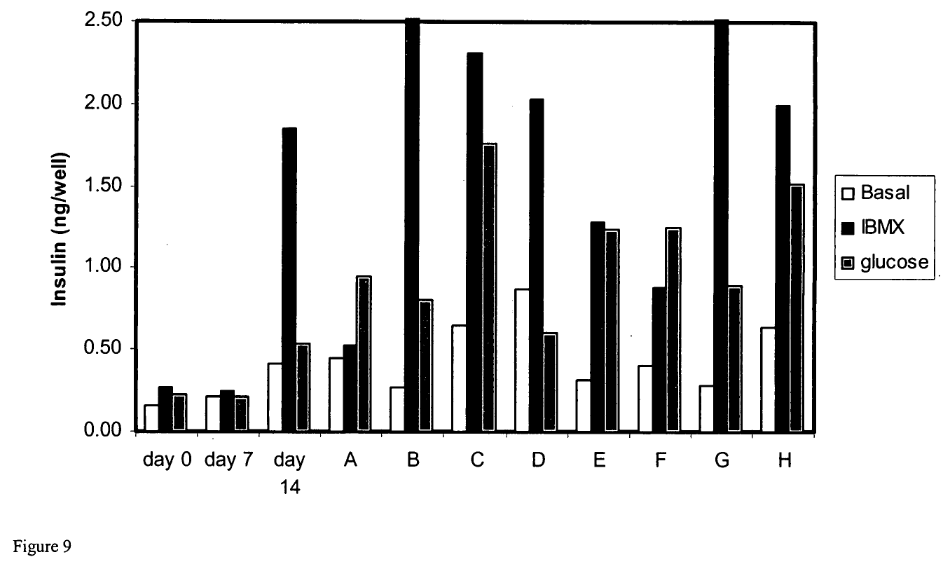

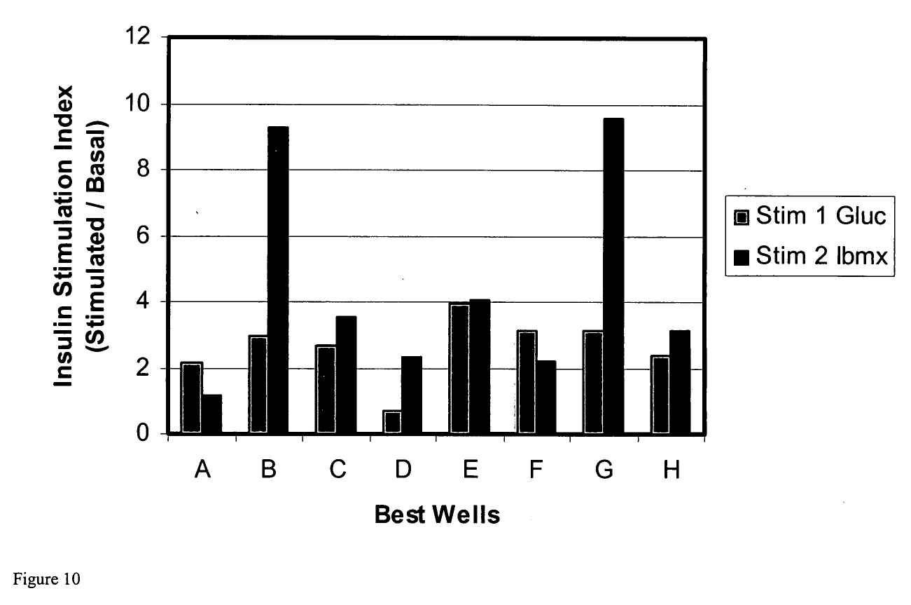

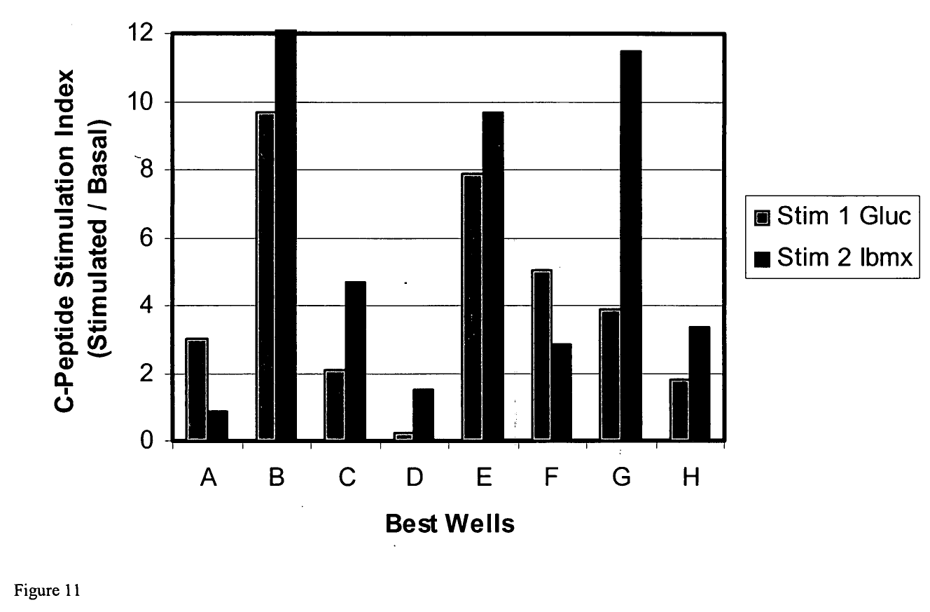

Examining four different experiments using cells from four different donors show that there was donor to donor variation in these experiments. The eight best wells were determined by the analyses from all donors. A comparison of each of these individual donors follows. In donor #2212 the insulin release from basal versus IBMX stimulation are shown in FIG. 3. Compared with day 0, each of the best wells had significant increase in stimulated insulin except for wells A, D and E. All of the wells from this donor had somewhat high basal insulin. Expressing the results as stimulation index (FIG. 4) show that best wells B, C, F, G and H had the highest responses. Examining the results of donor #2278 the insulin release from basal, high glucose or IBMX show a significant difference over the control wells at day 0, 7 and 14 (FIG. 5). This donors best wells all had very high basal insulin for reasons that were unclear resulting in a low stimulation index for all the best wells (FIG. 6). Examining the results of donor #3023, the insulin release from basal, high glucose or glucose and IBMX from the best wells were compared with day 0, 7 and 14 controls (FIG. 7). With lower basal insulin release for this donor, essentially all the best wells had significant stimulated insulin release. Calculating the stimulation index (FIG. 8) these also showed significant release from the best wells. However, there were differences in the insulin release with the best wells, each with different factor combinations. Some showed IBMX release higher than glucose (expected) and others showed glucose higher than glucose plus IBMX. This suggests the different combinations in these wells result in insulin producing cells with different capabilities. Examining the results for the fourth donor (3036), the basal insulin levels were low with significant stimulated insulin release after glucose or glucose plus IBMX challenge. (FIG. 9). Looking back at the wells in FIG. 2, these best wells are clearly better than most of the responses, as is the case from the other donors. Examination of the stimulation index (FIG. 10) show that the insulin producing cells generated from this donor gave higher responses with glucose plus IBMX compared to glucose alone. These supernatants were assayed for c-peptide content as shown in FIG. 11 as stimulation indices for c-peptide release.

In summary, these results demonstrate marked differences between wells containing different combinations of growth and differentiation factors as well as donor to donor variation. The selected best wells are not the final answer and additional studies are required in order to define the optimal combinations.

Table 1 shows the growth factor composition of these best wells

TABLE 1
Composition of Media Resulting in Best Insulin Production

| | Conc. | | | | | | | | |
|---|---|---|---|---|---|---|---|---|---|
| Substance | (μg/ml) | A | B | C | D | E | F | G | H |
| Activin A | 0.0005 | | ● | ● | | ● | | | |
| Atrial Natriuretic Peptide | 0.1530 | ● | | | ● | ● | | ● | |
| Betacellulin | 0.0050 | | ● | ● | | | ● | | |
| Bone Morphogenic Protein (BMP-2) | 0.0050 | ● | ● | | ● | ● | ● | | |
| Bone Morphogenic Protein (BMP-4) | 0.0005 | | ● | | ● | | | | |
| C natriuretic peptide (CNP) | 0.1099 | | ● | ● | ● | | | | ● |
| Caerulein | 0.0300 | ● | ● | | ● | | | ● | ● |
| CCK8 | 0.0057 | | | | | ● | ● | | ● |
| CCK8 (26-33), amide, | 0.0250 | | ● | | | | ● | ● | |
| CGRP alpha | 0.1905 | | ● | | | ● | | | ● |
| Cholera Toxin B Subunit | 0.0125 | | | | ● | ● | ● | ● | ● |
| Corticosterone | 0.0020 | | | ● | | | ● | | |
| Dexamethasone | 0.0020 | | | ● | | ● | ● | ● | ● |
| DIF-1/Differanisole A | 0.3000 | | ● | ● | | ● | | | ● |
| DMF (n n dimethylformamide) | 0.0000 | ● | ● | ● | | | ● | | ● |
| DMSO (dimethylsulfoxide) | 0.0010 | ● | ● | | ● | | ● | | ● |
| EGF | 0.0050 | | ● | | ● | | | | |
| Endothelin 1 | 0.5000 | ● | ● | ● | ● | | ● | | ● |
| Exendin 4 | 0.0210 | ● | ● | | ● | | | | ● |
| FGF acidic (aFGF = FGF1) | 0.0025 | ● | ● | ● | | ● | ● | ● | ● |
| FGF7 (KGF) | 0.0025 | ● | ● | | | ● | ● | ● | |
| FGFb (=FGF2) | 0.0025 | ● | ● | | | | | ● | |
| Gastrin I Human | 0.0000 | | | | | | | ● | |
| GLP-1 (7-36) amide, human (Glucagon-Like Peptide 1) | 0.0330 | | | | | ● | | ● | |
| Glucose | 1.0800 | ● | | ● | ● | ● | | ● | ● |
| Growth Hormone (somatotropin) | 0.0250 | | | | ● | | ● | | ● |
| GRP (Gastrin Releasing Peptide) | 0.1430 | ● | ● | | | | ● | ● | |
| Hepatocyte Growth Factor (HGF) | 0.0025 | | | | | ● | ● | ● | |
| IGF-1, recombinant human | 0.0025 | ● | | ● | ● | | | ● | |
| IGF-2, recombinant human | 0.0025 | ● | | | ● | | ● | ● | ● |
| Insulin | 9.5000 | ● | ● | ● | ● | ● | ● | ● | ● |
| Lactogen, from human placenta | 0.0500 | | ● | ● | | ● | | | |
| Laminin | 2.2500 | ● | | ● | | ● | ● | | ● |
| Leu-Enkephalin | 0.0030 | | | ● | | | ● | ● | |
| LIF, human (leukemia inhibitory factor) | 0.0025 | | | ● | | ● | | | |
| Met-Enkephalin | 0.0030 | | | | ● | ● | | | ● |
| n Butyric Acid, Sodium Salt | 4.5400 | ● | ● | ● | | | | ● | ● |
| Nerve Growth Factor, human (beta NGF) | 0.0025 | ● | ● | | ● | | ● | | ● |
| Nictotinamide | 610 | ● | | ● | ● | ● | ● | ● | ● |
| PDGF AA + PDGF BB MIX | 0.0050 | ● | ● | | | ● | | | ● |
| PIGF (Placental GF, human) | 0.0025 | ● | | | | | | | ● |
| Progesterone | 0.0030 | | ● | | | | ● | ● | ● |
| Prolactin | 0.0012 | ● | | | | | ● | ● | |
| pT II RP (Parathyroid Hormone Related Peptide) | 0.2060 | ● | | ● | | ● | | ● | ● |
| Putrescine Dihydrochloride Gamma-Irradiated Cell Culture | 0.0001 | ● | | | | ● | ● | | ● |
| REG1, Novocell Peptide Mimetic | 0.0326 | | | ● | | ● | ● | | |
| Retinoic Acid (Vitamin A) | 0.0250 | ● | ● | | ● | ● | ● | ● | ● |
| Selenium (Selenious Acid, Na salt) | 0.0250 | | | | | | ● | ● | |
| Sonic Hedgehog (mouse, recombinant) | 0.0250 | ● | ● | ● | ● | ● | ● | ● | |
| Substance P (full length) (H1875 is frag 1-4) | 5 | | | ● | ● | ● | ● | | |
| Superoxide Dismutase (SOD) | 5 IU/ml | | ● | | ● | ● | | | |
| TGF alpha | 0.0010 | | | ● | ● | ● | | | ● |
| TGF B1 | 0.0005 | | ● | | | | ● | | ● |
| TGF beta sRII (soluble receptor type 2) | 0.0050 | | ● | | | ● | ● | ● | |
| transferrin | 2.7500 | ● | ● | | | ● | | ● | |
| Triiodothyranine (T3) | 0.0335 | ● | | ● | | | | | ● |
| Trolox (soluble Vitamin E) | 0.6250 | | | ● | | | ● | ● | ● |
| Trypsin Inhibitor, soybean (type I-S) | 0.5000 | | ● | ● | ● | ● | ● | ● | ● |
| Vasoactive Intestinal Peptide (VIP) | 0.0665 | | ● | ● | | ● | | ● | ● |
| VEGF | 0.0025 | ● | | ● | | ● | | | ● |

### Example 4

Culture Media Analysis of Cells Cultured in Alginate

Statistical analysis of the insulin content of the supernatants generated by 3 donors, produced in example 3 from the combinatorial array, resulted in a list of positive and negative effectors influencing insulin production and cell growth, as well as, consistently good combinations.

Growth and differentiation factors that had a potential positive effect on the conversion of stem cells into insulin producing cells, as identified by this combinatorial system, are: Betacellulin, BMP-2, Caerulein, CCK8 sulfated, Cholera Toxin B Subunit, CNP, Corticosterone, DMF, DMSO, EGF, Exendin 4, FGF-1, Glucose, GRP, IGF-1, IGF-2, Insulin, KGF, Laminin, Leu-Enkephalin, Met-Enkephalin, NGF beta, Nictotinamide, PDGF AA.BB, pTHRP, Selenium, SHH, Substance P, TGF beta sRII, Transferrin, vEGF, VIP.

Growth and differentiation factors that had a potential negative effect on the conversion of stem cells into insulin producing cells, as identified by this combinatorial system, are: Activin A, ANP, BMP-4, CCK8 amide, CGRP alpha, Dexamethasone, DIF-1, Endothelin 1, FGF-2, Gastrin I, GH, GLP-1, HGF, Lactogen, LIF, n Butyric Acid, P1GF, Progesterone, Prolactin, Putrescine, REG-1, Retinoic Acid, SOD, Soybean Trypsin Inhibitor, T3, TGF alpha, TGF beta 1, Trolox

### Example 5

Sequential Culture of Stem Cells in Adherent Followed by Adherent Culture.

Stem cells, generated by a 6-12 day adherent culture on collagen coated plates in PCM, were cultured on collagen coated plates for an additional 8 days in basal medium supplemented with combinations of 60 growth factors in a 120 combinatorial array. Alternatively cells were removed from the collagen coated plates after the first culture period and replated onto fresh culture plates then cultured for an additional 8 days in basal medium supplemented with combinations of 60 growth factors in a 120 combinatorial array. At the end of the culture period cells were subjected to a 24 hr challenge with basal medium or 20 mM glucose. Supernatants were harvested and analyzed for insulin or C-peptide content using an ELISA. Cells were washed and lysed and the DNA content per well determined using a picogreen assay

Data from wells that constitutively produced insulin or induced to produce insulin glucose stimulation from 3 independent preparations were subjected to statistical analysis and “best wells” identified. The composition of growth factors present in the top four “best wells” is shown in Table 2.

TABLE 2
Composition of Media Resulting in Best Insulin Production

| | Conc. | | | | |
|---|---|---|---|---|---|
| Substance | (μg/ml) | I | J | K | L |
| Activin A | 0.0005 | | ● | | |
| Atrial Natriuretic Peptide | 0.1530 | | ● | ● | ● |
| Betacellulin | 0.0050 | ● | | ● | |
| Bone Morphogenic Protein (BMP-2) | 0.0050 | | ● | | |
| Bone Morphogenic Protein (BMP-4) | 0.0005 | | | ● | |
| C natriuretic peptide (CNP) | 0.1099 | ● | | ● | |
| Caerulein | 0.0300 | ● | | | ● |
| CCK8 sulphated | 0.0057 | | ● | ● | |
| CCK8 (26-33), amide, | 0.0250 | ● | | ● | ● |
| CGRP alpha | 0.1905 | ● | ● | ● | |
| Cholera Toxin B Subunit | 0.0125 | ● | ● | | ● |
| Corticosterone | 0.0020 | ● | | ● | |
| Dexamethasone | 0.0020 | ● | ● | | ● |
| DIF-1/Differanisole A | 0.3000 | ● | ● | ● | |
| DMF (n n dimethylformamide) | 0.0000 | | | ● | |
| DMSO (dimethylsulfoxide) | 0.0010 | ● | | | |
| EGF | 0.0050 | ● | | | |
| Endothelin 1 | 0.5000 | | | ● | |
| Exendin 4 | 0.0210 | | | | |
| FGF acidic (aFGF = FGF1) | 0.0025 | | ● | | ● |
| FGF7 (KGF) | 0.0025 | ● | | ● | ● |
| FGFb (=FGF2) | 0.0025 | | | | ● |
| Gastrin I Human | 0.0000 | | | ● | ● |
| GLP-1 (7-36) amide, human (Glucagon-Like Peptide 1) | 0.0330 | ● | ● | ● | ● |
| Glucose | 1.0800 | | ● | ● | ● |
| Growth Hormone (somatotropin) | 0.0250 | | | | |
| GRP (Gastrin Releasing Peptide) | 0.1430 | | | | ● |
| Hepatocyte Growth Factor (HGF) | 0.0025 | | ● | ● | ● |
| IGF-1, recombinant human | 0.0025 | ● | | | ● |
| IGF-2, recombinant human | 0.0025 | ● | | ● | ● |
| Insulin | 9.5000 | ● | ● | ● | ● |
| Lactogen, from human placenta | 0.0500 | | ● | ● | |
| Laminin | 2.2500 | ● | ● | | |
| Leu-Enkephalin | 0.0030 | ● | | | ● |
| LIF, human (leukemia inhibitory factor) | 0.0025 | ● | ● | ● | |
| Met-Enkephalin | 0.0030 | ● | ● | ● | |
| n Butyric Acid, Sodium Salt | 4.5400 | ● | | | ● |
| Nerve Growth Factor, human (beta NGF) | 0.0025 | ● | | ● | |
| Nictotinamide | 610 | | ● | ● | ● |
| PDGF AA + PDGF BB MIX | 0.0050 | | ● | | |
| PIGF (Placental GF, human) | 0.0025 | ● | | ● | |
| Progesterone | 0.0030 | ● | | | ● |
| Prolactin | 0.0012 | | | ● | ● |
| pT II RP (Parathyroid Hormone Related Peptide) | 0.2060 | ● | ● | ● | ● |
| Putrescine Dihydrochloride Gamma-Irradiated Cell Culture | 0.0001 | | ● | ● | |
| REG1, Novocell Peptide Mimetic | 0.0326 | ● | ● | | |
| Retinoic Acid (Vitamin A) | 0.0250 | ● | ● | ● | ● |
| Selenium (Selenious Acid, Na salt) | 0.0250 | | | | ● |
| Sonic Hedgehog (mouse, recombinant) | 0.0250 | ● | ● | ● | ● |
| Substance P (full length) (H1875 is frag 1-4) | 5 | ● | ● | | |
| Superoxide Dismutase (SOD) | 5 IU/ml | ● | ● | | |
| TGF alpha | 0.0010 | ● | ● | ● | |
| TGF B1 | 0.0005 | | | | |
| TGF beta sRII (soluble receptor type 2) | 0.0050 | ● | ● | ● | ● |
| transferrin | 2.7500 | ● | ● | | ● |
| Triiodothyronine (T3) | 0.0335 | | | | |
| Trolox (soluble Vitamin E) | 0.6250 | | | | ● |
| Trypsin Inhibitor, soybean (type I-S) | 0.5000 | | ● | | ● |
| Vasoactive Intestinal Peptide (VIP) | 0.0665 | | ● | | ● |
| VEGF | 0.0025 | | ● | | |

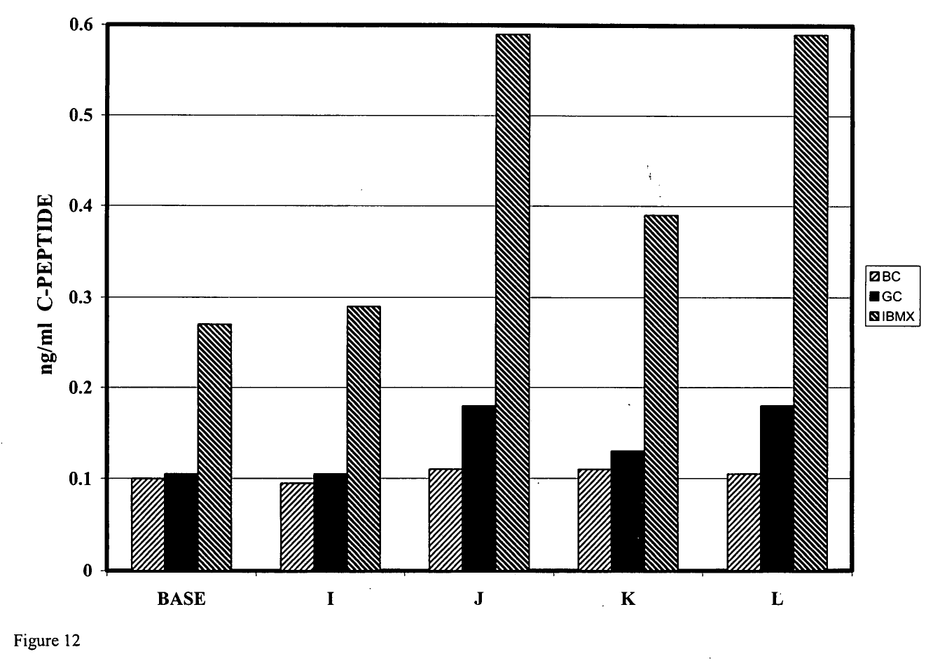

FIG. 12 presents the c-peptide results from this experiment showing release from basal, glucose and glucose plus IBMX stimulations showing positive responses. Ranges of insulin and DNA concentration were detected in the samples harvested from individual wells demonstrating that this is a feasible method for screening growth and differentiation factor combinations for their effect in the growth and differentiation of pancreatic cell derived stem cells

### Example 6

Further Optimization of the 120 Combinatorial Array.

Data presented in previous examples identified “best wells”, in terms of induced insulin production, or total insulin production. The ingredients present in the “best media” can then undergo a second tier screen to simplify and better define a minimal number of factors that induce the production of insulin-producing cells from stem cells. Alternatively, the positive effectors (Example 4) can undergo a second tier screening to achieve the same result.

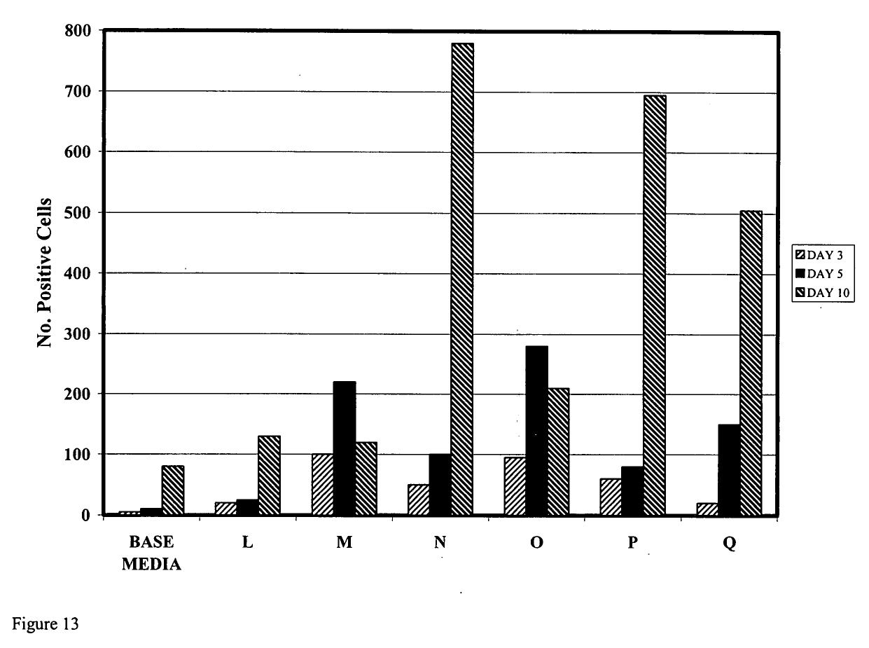

In this example, thirty components of media “L” were arrayed into a 60 factor array. Stem cells, generated by a 7 day adherent culture on collagen were placed into screening conditions for an additional 3, 5 or 10 days. At each time point, cells were fixed and processed for immunohistochemistry using a proinsulin-specific antibody. The number of proinsulin-positive cells was counted using automated image analysis. The number of pro-insulin positive cells using media M, N, O, P, and Q on days 3, 5 and 7 is shown in FIG. 13. These media are the most promising of the second tier screen. In the figure, they are compared to media “L”, a promising media from the 60 factor array. In conclusion, this example shows that the 60 factor combinatorial array can be refined and improved.

### Example 7

### Gene-Chip Studies (DNA Oligo Microarray)

The use of a “gene chip” (BD Atlas array) allows us to measure the relative expression levels (mRNA levels) of 8,000 genes. This method can be used to “fingerprint” or identify cell types. The analysis of mRNA expression in differentiating pancreatic cells potentially identifies genes that are involved in the transdifferentiation process. This type of comparison will allow us to compare starting pancreatic cells to the intermediary stem cell, intermediary stem cells to hormone-producing cells, and this final product to normal human pancreatic islets.

The utilization of such technology to produce a “finger print” of the gene expression patterns of the different cell types' found in the human pancreas would serve two critical functions. Perhaps the most important function of such an analysis would be to clearly define the gene expression profile of cells generated during the transdifferentiation process and thus define, on the molecular level, the unique characteristics of these cells. The second function of such an analysis would be to provide tools to improve our research methods. The analysis would give us insights into the mechanisms of how the insulin-producing phenotype is regulated. Knowledge of cell surface markers would facilitate rapid cell identification as well as provide the means to sort desirable cells from “undesirable” cells. Information of the cell signaling molecules and transcription factors present on these cells will facilitate the identification of growth factors that may be required to more efficiently complete expansion and transdifferentiation of the starting material into cells capable of producing insulin in a similar manner to naturally occurring beta cells. While there is some information on gene expression and phenotype of pancreatic cells available in publicly available literature and reports, much of it relates to non-human animal models, or embryonic development. These gene chip studies are specific to our applications and discoveries.

Tables 3 and 4 show the result of two islet-depleted, human pancreatic cell preparations that were compared after 7 days of culture in adherent culture in PCM. RNA was isolated by standard methods and screened in comparative micro arrays. While the two preparations were cultured under identical conditions, one preparation was judged to be “excellent”, while the other was judged to be “OK” (by the criteria of its subsequent ability to produced c-peptide). Most of the genes expressed in these cultures will be the same, but there will be some genes that are differentially expressed. Some differences will be donor specific (e.g. differences in MHC markers), while others may give us insights into the genes that are determinative in “excellent” versus “OK” results.

Examination of the 8,000 genes expressed by each of the different preparations result in an extensive list that is too long to include. Table 3 summarizes those genes that we believe may be particularly useful to our studies and objectives for obtaining new insulin-producing cells. Some of these genes are mechanistically important to the differentiation process, while others are correlative and possibly predictive of successful stem cell formation. Table 4 is a compilation of about 90 “strongly expressed” messages (signal strength of 10-100% of maximal). The strongly expressed messages may be particularly useful in identifying surface markers that can be used to identify and sort the different cell populations (acinar vs islet or successfully differentiated vs poorly differentiated). Again, the complete list of “strongly expressed” genes is extensive and an abbreviated version is presented.

TABLE 3
A summary of important/useful genes expressed at comparatively different levels in the
“excellent” prep (2071) versus the “OK” prep (2078).

| | | Ratio prep | | |
|---|---|---|---|---|
| Line # | Gene | 2078:2071 | Genbank | Short summary |

Genes expressed at a higher level in the “excellent” prep.

| | | | | |
|---|---|---|---|---|
| 574 | hairless (mouse) | Down 5.4 | AF039196 | A transcription factor, found in many different tissues |
| | homolog | | | with highest expression in brain. May function as a |
| | | | | specific repressor. Difference between two preps is |
| | | | | great, higher expression in “good” prep. |
| 1237 | sine oculis | Down 2.6 | NM_005413 | A homeobox gene that has been studied in eye |
| | homeobox | | | development; also expressed in adult (fully |
| | (Drosophila) | | | differentiated eye tissues). Activates Pax6 expression! |
| | homolog 3 | | | (Pax6 is a mature beta cell marker). No publications |
| | | | | regarding sine oculis in pancreatic literature. |
| 16 | 7-60 protein | down 2.5 | NM_007346 | Receptor for opioid growth factor, Met(5)-enkephalin |
| | | | | (a factor present in the MFA). Ligand is an inhibitory |
| | | | | peptide that modulates cell proliferation and tissue |
| | | | | organization during development, cellular renewal, |
| | | | | cancer, wound healing, and angiogenesis. |
| 234 | CDC37 (cell | down 2.8 | U63131 | Positive regulator of cell cycle progression through |
| | division 37, S. | | | interactions with CDK4. May also be a component of |
| | cerevisiae, | | | a complex that regulates NF-kappa B. |
| | homolog) | | | |

Genes expressed at a higher level in the “OK” prep (2078)

| | | | | |
|---|---|---|---|---|
| 866 | neurogenic | up 2.6 | NM_006160 | A helix-loop-helix transcription factor known to |
| | differentiation 2 | | | mediate neuronal differentiation. Closely related to |
| | (ND2) | | | NeuroD1. (aka “Beta cell E-box transactivator” or |
| | | | | “Beta 2”). Role for ND1 well established in mouse |
| | | | | model, role of ND2 not determined. Is ND2 |
| | | | | predominant acinar form? Predominant |
| | | | | transdifferentiating form? |
| 935 | pancreatitis- | up 4.1 | D13510 | An acinar protein. Abundance is normally very low, |
| | associated protein | | | but very high in pancreatitis. It is also a marker for |
| | | | | some liver cancers. Function? Is expression induced |
| | | | | during transdifferentiation? |
| 489 | G protein-coupled | up 3.1 | NM_005682 | Has similarity to some secretin-like receptors and has |
| | receptor 56 | | | a mucin-like domain. Present in a wide range of |
| | | | | tissues. Highest levels in the smaller, more actively |
| | | | | secreting follicles of human thyroid. Marker for |
| | | | | undifferentiated acinar? |
| 1115 | retinoic acid | up 3.2 | NM_002889 | Retinoids exert potent growth inhibitory and cell |
| | receptor responder | | | differentiation activities. These effects are mediated |
| | (tazarotene | | | by specific nuclear receptor proteins that are members |
| | induced) 2 | | | of the steroid and thyroid hormone receptor |
| | | | | superfamily of transcriptional regulators. Marker for |
| | | | | undifferentiated acinar? |

TABLE 4
A summary of potentially important and/or useful genes expressed at high levels in both cell
preparations after 7 days in culture.

| | | | |
|---|---|---|---|
| Line # | Gene | Genbank | Short summary |

Enzymes & cofactors

| | | | |
|---|---|---|---|
| 122 | ATPase Ca++ | L20977 | Membrane Ca++ pump, highly restricted tissue distribution, well |
| | transporting | | characterized in cochlear outer hair cells and spiral ganglion. |
| | plasma | | Expression is strong. Good acinar membrane marker? |
| | membrane 2 | | |
| 387 | Dual-specf. Tyr. | NM_004714 | Regulates nuclear functions? Implicated in postembryonic |
| | Phosph regulated | | neurogenesis. Also, enables colon carcinoma cells to survive |
| | protein | | under certain stress conditions |
| 657 | Inhib of kappa | NM_003639 | Kinase that activates the enhancer of NF-kappa-B activation; it |
| | light polypeptide | | would play a role in activating the response to inflammatory |
| | enhancer kinase | | cytokines. Perhaps a role in differentiating cells? |
| 659 | Inositol | NM_001567 | INPPL or SHIP2 may play a significant role in regulation of |
| | polyphosphate | | P13K signaling by growth factors and insulin. Primary defect in |
| | phosphatase-like | | KO mouse shows that SHIP2 is a potent negative regulator of |
| | | | insulin signaling and insulin sensitivity in vivo. Important |
| | | | regulator of growth factor signaling in our system? |
| 824 | MAP kinase | NM_004579 | Found in many tissues, participates in B-cell differentiation. |
| | kinase kinase | | |
| | kinase 2 | | |
| 1228 | Sialyl transferase 8 | NM_003034 | Modifies NCAM with polysialic acid. Involved in modifying cell |
| | | | adhesion molecules in our system? |

DNA, transcription factors and developmental genes.

| | | | |
|---|---|---|---|
| 196 | Cardiac-specific | NM_004387 | aka NKX2-5. Homeobox-containing genes are essential for |
| | homeo box | | tissue differentiation, as well as determining the temporal and |
| | | | spatial patterns of development. This one has been characterized |
| | | | in terms of heart formation. Mouse pancreas researchers have |
| | | | focused on nkx2.2 and nkx6.1 |
| 200 | Cartilage paired | NM_006982 | Function unknown in humans. In mouse, necessary for survival |
| | class | | of the forebrain mesenchyme. Mutations lead to acrania and |
| | homeoprotein | | meroanencephaly. |
| 213 | C/EBP alpha | U34070 | Regulates differentiation in a number of cell types. Has also |
| | | | been shown to inhibit cyclin-dependent kinases and cause growth |
| | | | arrest. |
| 214 | C/EBP beta | NM_005194 | Regulates differentiation in a number of cell types. Required for |
| | | | a normal proliferative response. |
| 933 | Paired | NM_002653 | Bicoid class of homeodomain proteins. Members of this family |
| | homeodomain | | are involved in organ development, left-right asymmetry. Also |
| | transcription | | acts as a transcriptional regulator in some adult tissues (e.g. |
| | factor | | prolactin gene). In developmental models, Pitx2 is directly |
| | | | initiated by Nodal signaling and is subsequently maintained by |
| | | | Nkx2. If it is maintained by Nkx2, may be present in maturing |
| | | | alpha and beta cells. |
| 995 | POU domain, | NM_002699 | AKA Oct-6. Involved in nerve development and regeneration; |
| | class 3 | | other developmental roles? Oct 4 plays a role in mouse |
| | transcription | | pancreatic development. |
| | factor | | |
| 1285 | Spi | NM_003120 | Related to ets. Essential for the development of myeloid and B- |
| | | | lymphoid cells |
| 1347 | Transcription | NM_003206 | A basic helix-loop-helix transcription factor, In adults, expressed |
| | factor 21 | | in lung, kidney, heart, placenta and pancreas. In embryos, |
| | | | essential for the development of the coronary vasculature and |
| | | | organs containing epithelial-lined tubular structures. May |
| | | | represent a point of regulatory convergence between a number of |
| | | | transcription factors. |

Growth factor and related genes

| | | | |
|---|---|---|---|
| 90 | Anti Mullerian | NM_000479 | Anti-Mullerian hormone is a member of the TGF-beta and |
| | Hormone | | inhibin gene family. Mediates male sexual differentiation: |
| | | | Causes the regression of Mullerian ducts which would otherwise |
| | | | differentiate into the uterus and fallopian tubes. Unknown |
| | | | function in adults. |
| 935 | Pancreatitis- | D13510 | Expression is low in normal acinar cells but very high in |
| | associated protein | | pancreatitis. Also expressed by epithelial cells of the small |
| | | | intestine and some liver cancers. Function? |
| 197 | Cardiotropin 1 | NM_001330 | Family of cytokines that includes LIF, ciliary neurotrophic factor |
| | | | (CNTF), oncostatin M, interleukin 6 and 11 Required for |
| | | | motorneuron development, promotes motorneuron survival. |
| 808 | Midkine neurite | NM_002391 | Exhibits neurite outgrowth-promoting activity and plays a role in |
| | growth promoting | | nervous system development and/or maintenance. Expression |
| | factor 2 | | believed to be very low, except for a short period during |
| | | | development |

Receptor and signal transduction related

| | | | |
|---|---|---|---|
| 81 | Angiotensin | NM_004835 | Angiotensin is an important effector controlling blood pressure |
| | receptor 1B | | and volume in the cardiovascular system. These receptors are |
| | | | also found in the exocrine, endocrine and vascular cells of the |
| | | | pancreas. Immunostaining to AR is predominantly in the |
| | | | endothelia of the blood vessels and the epithelia of the pancreatic |
| | | | ductal system and weakly in ascini. |
| 550 | Growth hormone | NM_005310 | Diverse family important in tyrosine kinase signaling. |
| | receptor-bound | | Homologous to ras-GAP. In some models, involved in metastatic |
| | protein 7 | | progression |
| 554 | Growth hormone | NM_004122 | GRS and GH releasing factor have the reciprocal effect of |
| | secretagogue | | somatostatin on growth hormone release from the pituitary (see |
| | receptor | | next). This G-protein coupled receptor can also bind ghrelin. |
| | | | Would have expected this marker on endocrine cells. |
| 1277 | Somatostatin | NM_001051 | Somatostatin acts at many sites to inhibit the release of many |
| | receptor 3 | | hormones and other secretory proteins. The biological effects of |
| | | | somatostatin are probably mediated by G protein-coupled |
| | | | receptors that are expressed in a tissue-specific manner. SSTR3 |
| | | | is expressed in highest levels in brain and pancreatic islets. |
| 1056 | Protein tyrosine | NM_002850 | Receptor-type PTP. A signaling molecule that may regulate |
| | phosphatase | | growth and differentiation. This PTP has been also implicated in |
| | | | the control of adult nerve repair. |
| 426 | Ephrin A5 | U26403 | Binds to members of the EPH group of receptor tyrosine kinases. |
| | | | May be involved in axon guidance. Ephrin and its receptor may |
| | | | shift the cellular response from repulsion to adhesion. |
| 172 | Butyrate response | X79067 | Induced by various agents: phorbol ester TPA, EGF, etc. May |
| | factor 1 (EGF | | mediate rapid degradation of cytokine (AU-rich) mRNA. Marker |
| | response factor 1) | | for activation/differentiation? |

Cell surface or structural genes

| | | | |
|---|---|---|---|
| 47 | Adenomatous | NM_005883 | Located in both the membrane/cytoskeletal and the nuclear |
| | polyposis | | fraction, ubiquitously expressed. APC interacts with catenins, |
| | coli-like | | and through these, with E-cadherin. May regulate transmission of |
| | | | the contact inhibition signal into the cell, or may regulate |
| | | | adhesion. The former is more consistent with mutated APC's |
| | | | early role in tumorigenesis. Depending on what proteins are |
| | | | members of the APC complex, APC may participate in cell cycle |
| | | | progression, developmental pathways cell morphology or |
| | | | neuronal function. |
| 95 | Aquaporin 5 | NM_001651 | Aquaporins are water channel proteins. Aq5 is known to play a |
| | | | role in the generation of saliva, tears and pulmonary secretions. |
| | | | Marker for duct epithelium? |
| 220 | CD151 antigen | NM_004357 | Member of the transmembrane 4 superfamily, aka tetraspanin |
| | | | family. A cell surface glycoprotein that is known to complex |
| | | | with integrins and other tetraspanins. The complexes are cell- |
| | | | attachment sites for binding to basement membranes. The |
| | | | proteins mediate signal transduction events in the regulation of |
| | | | cell development, activation, growth and motility. |
| 221 | CD3 E antigen | NM_000733 | CD3 epsilon is one of the T-cell antigen receptor complex |
| | | | subunits. CD3-E is a signal transducing component that may be |
| | | | particularly important in instructing pre T Cell lineage |
| | | | commitment. Role in pancreatic lineage commitment? |
| 303 | Cofilin | NM_005507 | A widely distributed intracellular actin-modulating protein that |
| | | | depolymerizes filamentous actin and inhibits the polymerization |
| | | | of monomeric actin. |
| 465 | Ficolin | NM_003665 | Characterized as a serum protein. Has calcium-independent |
| | | | lectin activity but unlike other family members, it does not bind |
| | | | fibronectin or elastin. |
| 669 | Integrin, alpha 3 | NM_002204 | Cell surface adhesion molecule, integral membrane protein; |
| | | | interacts with many extracellular-matrix proteins |
| 690 | Junction | M23410 | A major cytoplasmic protein that occurs in a soluble and a |
| | plakoglobin | | membrane-associated form and in adhering junctions |
| | | | (desmosomes and intermediate junctions) |
| 694 | Keratin 7 | x03212 | Expression patterns of CK19 have been very useful for us |
| | Keratin 17 | NM_000422 | already; the other keratins may also be useful. |
| | Keratin 8 | M34225 | |
| 872 | Neuronal thread | NM_014486 | NTP is one of the proteins expressed during growth and |
| | protein | | sprouting of neuronal cells. Expressed during |
| | | | transdifferentiation to neuroendocrine cells? |
| 1008 | Profilin 1 | NM_005022 | A ubiquitous actin monomer-binding protein. Regulates actin |
| | | | polymerization in response to extracellular signals. |
| 1187 | S100 Ca++ | NM_005620 | Family of proteins localized in the cytoplasm and/or nucleus of a |
| | binding protein | | wide range of cells. Involved in the regulation of cell cycle |
| | | | progression and differentiation. Remarkably elevated in |
| | | | colorectal cancers compared to normal mucosa. |
| 1299 | Stratifin | AF029082 | Diffusely distributed in the cytoplasm. Most abundant in tissues |
| | | | enriched in stratified keratinizing epithelium. Mediates signal |
| | | | transduction by binding to phosphoserine-containing proteins. |
| | | | Induced in response to DNA damage, and causes cells to arrest in |
| | | | G2 AKA 14-3-3-sigma |
| 1335 | Thymosin beta | M92381 | An actin-sequestering protein |
| 1435 | Wishot Aldrich | NM_003387 | Involved in transduction of signals from receptors on the cell |
| | interacting protein | | surface to the actin cytoskeleton. Induces actin polymerization. |

Unknown function

| | | | |
|---|---|---|---|
| 829 | Mucin 6 | U97698 | A large glycoprotein that is thought to play a major role in |
| | | | protecting the gastrointestinal tract from acid, proteases, |
| | | | pathogenic microorganisms and mechanical trauma. |
| 1257 | Small pro-rich | NM_006945 | Keratinocyte differentiation marker. Function? |
| | protein | | |

What is claimed is:

1. A method of converting differentiated non-hormone producing pancreatic cells into differentiated hormone-producing cells, comprising:
a) culturing said differentiated non-hormone producing pancreatic cells in a first cell culture system with a first cell culture medium comprising a basal medium, with or without serum, and with or without growth factors;
under conditions which provide for converting said differentiated non-hormone producing pancreatic cells into stem cells; and
b) culturing said stem cells in a second cell culture system with a second cell culture medium comprising at least one compound selected from Group A, wherein the compounds of Group A are selected from the group consisting of:
Betacellulin, Activin A, BMP-2, TGF-β SRII, DMSO, Sonic Hedgehog, Laminin, Met-Enkephalin, DMF, and Cholera Toxin A;
and at least one compound selected from Group B, wherein the compounds of Group B are selected from the group consisting of:
Activin A, Atrial Natriuretic Peptide, Betacellulin, Bone Morphogenic Protein (BMP-2), Bone Morphogenic Protein (BMP-4), C natriuretic peptide (CNP), Caerulein, Calcitonin Gene Related Peptide (CGRP-ax), Cholecystokinin (CCK8-amide), Cholecystokinin octapeptide (CCK8-sulfated), Cholera Toxin B Subunit, Corticosterone (Reichstein's substance H), Dexamethasone, DIF-1, Differanisole A, Dimethylsulfoxide (DMSO), EGF, Endothelin 1, Exendin 4, FGF acidic, FGF2, FGF7, FGFb, Gastrin I, Gastrin Releasing Peptide (GRP), Glucagon-Like Peptide 1 (GLP-1), Glucose, Growth Hormone, Hepatocyte Growth Factor (HGF), IGF-1, IGF-2, Insulin, KGF, Lactogen, Laminin, Leu-Enkephalin, Leukemia Inhibitory Factor (LIF), Met-Enkephalin, n Butyric Acid, Nerve Growth Factor (β-NGF), Nicotinamide, n-n-dimethylformamide (DMF), Parathyroid Hormone Related Peptide (Pth II RP), PDGF AA+PDGF BB MIX, PIGF (Placental GF), Progesterone, Prolactin, Putrescine Dihydrochloride Gamma-Irradiated Cell Culture, REG1, Retinoic Acid, Selenium, Selenious Acid, Sonic Hedgehog, Soybean Trypsin Inhibitor, Substance P, Superoxide Dismutase (SOD), TGF-α, TGF-β sRII, TGF-β1, transferrin, Triiodothyronine (T3), Trolox, Vasoactive Intestinal Peptide (VIP), VEGF, Vitamin A, and Vitamin E;
under conditions which provide for differentiating said stem cells into hormone-producing cells.

2. The method of claim 1, wherein the second cell culture medium comprises at least two compounds selected from Group A and at least two compounds selected from Group B.

3. The method of claim 1, wherein the second cell culture medium comprises at least three compounds selected from Group A and at least three compounds selected from Group B.

4. The method of claim 1, wherein the second cell culture medium comprises at least four compounds selected from Group A and at least four compounds selected from Group B.

5. The method of claim 1, wherein the second cell culture medium comprises at least five compounds selected from Group A and at least five compounds selected from Group B.

6. The method of claim 1, wherein the second cell culture medium comprises at least six compounds selected from Group A and at least six compounds selected from Group B.

7. A method of culturing stem cells into differentiated hormone-producing cells, comprising culturing the stem cells in a cell culture system with a cell culture medium whereby said stem cells are differentiated into hormone-producing cells wherein said culture medium comprises basal medium without serum and at least one compound selected from Group A wherein the compounds of Group A are selected from the group consisting of: Betacellulin, Activin A, BMP-2, TGF-β SR11, DMSO, Sonic Hedgehog, Laminin, Met-Enkephalin, DMF, and Cholera Toxin A;
and at least one compound selected from Group B, wherein the compounds of Group B are selected from the group consisting of: Activin A, Atrial Natriuretic Peptide, Betacellulin, Bone Morphogenic Protein (BMP-2), Bone Morphogenic Protein (BMP-4), C natriuretic peptide (CNP), Caerulein, Calcitonin Gene Related Peptide (CGRP-α), Cholecystokinin (CCK8-amide), Cholecystokinin octapeptide (CCK8-sulfated), Cholera Toxin B Subunit, Corticosterone (Reichstein's substance H), Dexamethasone, DIF-1, Differanisole A, Dimethylsulfoxide (DMSO), EGF, Endothelin 1, Exendin 4, FGF acidic, FGF2, FGF7, FGFb, Gastrin I, Gastrin Releasing Peptide (GRP), Glucagon-Like Peptide 1 (GLP-1), Glucose, Growth Hormone, Hepatocyte Growth Factor (HGF), IGF-1, IGF-2, Insulin, KGF, Lactogen, Laminin, Leu-Enkephalin, Leukemia Inhibitory Factor (LIF), Met-Enkephalin, n Butyric Acid, Nerve Growth Factor (β-NGF), Nicotinamide, n-n-dimethylformamide (DMF), Parathyroid Hormone Related Peptide (Pth II RP), PDGF AA+PDGF BB MIX, PIGF (Placental GF), Progesterone, Prolactin, Putrescine Dihydrochloride Gamma-Irradiated Cell Culture, RE G1, Retinoic Acid, Selenium, Selenious Acid, Sonic Hedgehog, Soybean Trypsin Inhibitor, Substance P, Superoxide Dismutase (SOD), TGF-α, TGF-β sRII, TGF-β1, transferrin, Triiodothyronine (T3), Trolox, Vasoactive Intestinal Peptide (VIP), VEGF, Vitamin A, and Vitamin E.

8. The method of claim 7, wherein the cell culture medium comprises at least two compounds selected from Group A and at least two compounds selected from Group B.

9. The method of claim 7, wherein the cell culture medium comprises at least three compounds selected from Group A and at least three compounds selected from Group B.

10. The method of claim 7, wherein the cell culture medium comprises at least four compounds selected from Group A and at least four compounds selected from Group B.

11. The method of claim 7, wherein the cell culture medium comprises at least five compounds selected from Group A and at least five compounds selected from Group B.

12. The method of claim 7, wherein the cell culture medium comprises at least six compounds selected from Group A and at least six compounds selected from Group B.
